(19) United States
(12) Patent Application Publication
(10) Pub. No.: US 2015/0238149 A1
(43) Pub. Date: Aug. 27, 2015

(54) MAGNETIC RESONANCE IMAGING APPARATUS

(71) Applicants: KABUSHIKI KAISHA TOSHIBA, Tokyo (JP); TOSHIBA MEDICAL SYSTEMS CORPORATION, Otawara-shi (JP)

(72) Inventors: Shuhei NITTA, Tokyo (JP); Tomoyuki TAKEGUCHI, Kawasaki (JP); Taichiro SHIODERA, Tokyo (JP); Shigehide KUHARA, Otawara (JP)

(21) Appl. No.: 14/707,377

(22) Filed: May 8, 2015

Related U.S. Application Data
(63) Continuation of application No. PCT/JP2014/068510, filed on Jul. 10, 2014.

(30) Foreign Application Priority Data
Jul. 10, 2013 (JP) 2013-144948

(51) Int. Cl. A61B 5/00; A61B 5/113; A61B 5/0402; G01R 33/567

(52) U.S. Cl. CPC A61B 5/7289; G01R 33/5676; A61B 5/113; A61B 5/7278; A61B 5/0037; G01R 33/5673; A61B 5/0402; A61B 5/0044; A61B 2576/023

(57) ABSTRACT

A magnetic resonance imaging apparatus includes a controller and a deriving unit. The controller performs first imaging for acquiring first image data on a region including a target and the diaphragm, second imaging for acquiring, with application of motion detection pulses for detecting a respiratory phase, second image data including the target at a first respiratory phase and third image data including the target at a second respiratory phase different from the first respiratory phase, and third imaging for acquiring fourth image data. The deriving unit detects the position of the diaphragm from the first image data and derives a region to which the motion detection pulses are applied. In the performing of the second imaging, the controller detects a respiratory phase by the application of the detection pulses and controls timings at which the second image data and the third image data are acquired.

### CROSS-REFERENCE TO RELATED APPLICATIONS

This application is a continuation of PCT international application Ser. No. PCT/JP2014/068510 filed on Jul. 10, 2014 which designates the United States, incorporated herein by reference, and which claims the benefit of priority from Japanese Patent Application No. 2013-144948, filed on Jul. 10, 2013, the entire contents of all of which are incorporated herein by reference.

### FIELD

Embodiments relate to a magnetic resonance imaging apparatus.

### BACKGROUND

Magnetic resonance imaging is an imaging method in which nuclear spins of a subject who is positioned in a static magnetic field are magnetically excited by using RF (Radio Frequency) pulses of Larmor frequency and images are generated from the data of magnetic resonance signals that are generated in accordance with the excitation.

In conventional cardiac examination methods using magnetic resonance imaging, a standardization protocol is defined. For example, standardization protocol defines a flow in which, a scout view (body axis cross-section (axial view)), a sagittal cross-section (sagittal view), and a coronal cross-section (coronal view) are acquired, multi-slice views that are multiple body-axis cross-sections are then acquired, and cross-sectional views for diagnosis are then acquired.

The acquired multi-slice views are used to position the cross-sectional views for diagnosis. In order to perform the positioning accurately, multi-slice views are acquired while a subject is holding breath. The cross-sectional views for diagnosis are, for example, cross-sectional views based on the anatomical characteristics of the heart, such as a vertical long-axis view, horizontal long-axis view, two-chamber long-axis (2 chamber) view, three-chamber long-axis (3 chamber) view, four-chamber long-axis (4 chamber) view, and left-ventricle short-axis view.

### BRIEF DESCRIPTION OF DRAWINGS

FIG. 1 is a functional block diagram of a configuration of an MRI apparatus according to a first embodiment.

FIG. 2 is a flowchart of a procedure in the first embodiment.

FIG. 3 is a diagram of a respiratory phase setting GUI in the first embodiment.

FIG. 4 is a diagram for illustrating three-dimensional MR data in the first embodiment.

FIG. 5 is a flowchart of a procedure for detecting position information in the first embodiment.

FIG. 6 is a diagram for illustrating detection of position information in the first embodiment.

FIG. 7 is a diagram for illustrating deriving of various regions in the first embodiment.

FIG. 8 is a diagram for illustrating deriving of various regions in the first embodiment.

FIG. 9 is a diagram for illustrating acquisition of multi-slice views in the first embodiment.

FIG. 10 is a diagram for illustrating acquisition of multi-slice views in the first embodiment.

FIG. 11 is a diagram of a positioning GUI in the first embodiment.

FIG. 12 is a diagram for illustrating acquisition of multi-slice views in the first modification example of the first embodiment.

FIG. 13 is a diagram for illustrating acquisition of multi-slice views in the second modification example of the first embodiment.

FIG. 14 is a diagram for illustrating acquisition of multi-slice views in a second embodiment.

FIG. 15 is a diagram for illustrating acquisition of multi-slice views in the second embodiment.

FIG. 16 is a diagram of a positioning GUI in the second embodiment.

FIG. 17 is a diagram of a respiratory phase setting GUI in one of the other embodiments.

FIG. 18 is a diagram of a respiratory phase setting GUI in one of the other embodiments.

FIG. 19 is a diagram for illustrating acquisition of multi-slice views in one of the other embodiments.

FIG. 20 is a diagram of a respiratory phase setting GUI in one of the other embodiments.

FIG. 21 is a diagram of a hardware configuration of a computer that implements a calculator and a sequence controller according to the embodiments.

### DETAILED DESCRIPTION

A magnetic resonance imaging apparatus according to the embodiments include a controller and a deriving unit.

The controller performs first imaging for acquiring first image data, performs second imaging for acquiring second image data and third image data, and performs third imaging for acquiring fourth image data, the first image data being of a region including a target and a diaphragm, the second imaging and the third imaging being performed with application of motion detection pulses for detecting a respiratory phase, the second image data being of a region including the target at a first respiratory phase, the third image data being of a region including the target at a second respiratory phase being different from the first respiratory phase image. The deriving unit detects a position of the diaphragm from the first image data and on a basis of the detected position, derives a region to which the motion detection pulses are applied, wherein, in the performing of the second imaging, the controller detects the respiratory phase by the application of the motion detection pulses and, on a basis of the detected respiratory phase, controls timings at which the second image data and the third image data are acquired.

With reference to the drawings, magnetic resonance imaging apparatuses (hereinafter, “MRI apparatus” as appropriate) according to embodiments will be described below. Embodiments are not limited to the embodiments below. The content of descriptions given to each embodiment can basically be similarly used for other embodiments.

### First Embodiment

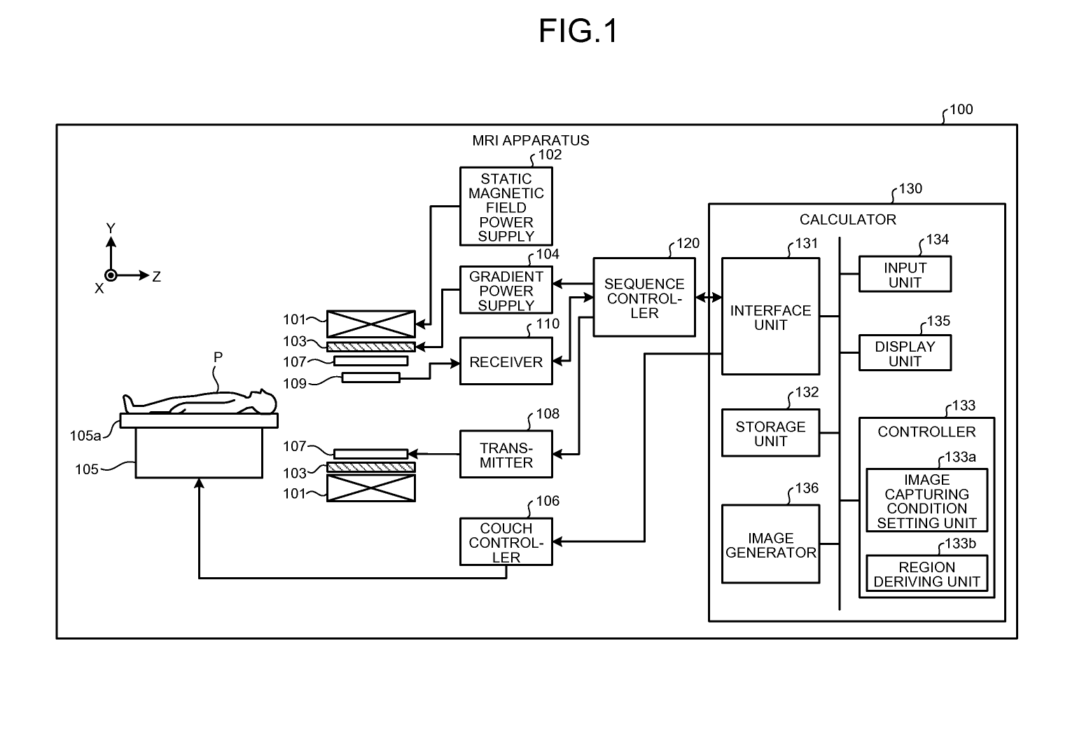

FIG. 1 is a functional block diagram of a configuration an MRI apparatus 100 according to the first embodiment. As shown in FIG. 1, the MRI apparatus 100 includes a static magnetic field magnet 101, a static magnetic field power supply 102, a gradient coil 103, a gradient power supply 104, a couch 105, a couch controller 106, a transmitter coil 107, a transmitter 108, a receiving coil 109, a receiver 110, a sequence controller 120, and a calculator 130. The MRI apparatus 100 does not include a subject P (e.g. a human body). The configuration shown in FIG. 1 is a mere example. For example, each unit of the sequence controller 120 and the calculator 130 may be configured integrally or separately.

The static magnetic field magnet 101 is a magnet that is formed into a hollow cylinder and that generates a static magnetic field in the internal space. The static magnetic field magnet 101 is, for example, a superconducting magnet that is magnetically excited when supplied with an electric current from the static magnetic field power supply 102. The static magnetic field power supply 102 supplies an electric current to the static magnetic field magnet 101. The static magnetic field magnet 101 may be a permanent magnet. In such a case, the MRI apparatus 100 is not required to include the static magnetic field power supply 102. The static magnetic field power supply 102 may be provided apart from the MRI apparatus 100.

The gradient coil 103 is a coil that is formed in a hollow cylinder and that is arranged on the inner side of the static magnetic field magnet 101. The gradient coil 103 is formed by combining three coils corresponding to X, Y and Z axes that are orthogonal to one another. Upon being supplied with an electric current individually from the gradient power supply 104, each of these three coils generates a gradient magnetic field where the magnetic field intensity changes along each of the X, Y and Z axes. The gradient magnetic fields of the X, Y and Z axes that are generated by the gradient coil 103 are, for example, a slice gradient magnetic field Gs, a phase encode gradient magnetic field Ge, and a readout gradient magnetic field Gr. The gradient power supply 104 supplies an electric current to the gradient coil 103.

The couch 105 includes a couchtop 105a on which the subject P is placed and, under the control of the couch controller 106, the couchtop 105a with the subject P placed thereon is caused to enter the hollow of the gradient coil 103 (imaging port). Generally, the couch 105 is set such that its longitudinal direction is parallel to the center axis of the static magnetic field magnet 101. Under the control of the calculator 130, the couch controller 106 drives the couch 105 to move the couchtop 105a along its longitudinal direction and vertical direction.

The transmitter coil 107 is arranged on the inner side of the gradient coil 103 and generates a high-frequency magnetic field when supplied with RF pulses from the transmitter 108. The transmitter 108 supplies RF pulses corresponding to the Larmor frequency that is determined by the target atom type and magnetic field intensity to the transmitter coil 107.

The reception coil 109 is arranged on the inner side of the gradient coil 103 and receives magnetic resonance signals (hereinafter, “MR signals” as required) that are emitted from the subject P due to the effects of the high-frequency magnetic field. Upon receiving MR signals, the reception coil 109 outputs the received MR signals to the receiver 110.

The transmitter coil 107 and the reception coil 109 are mere examples. It is satisfactory if they consist of any one of, or a combination of, a coil with only a transmission function, a coil with only a reception function, and a coil having a transmission and reception functions.

The receiver 110 detects the MR signals that are output from the reception coil 109 and generates MR data on the basis of the detected MR signals. Specifically, the receiver 110 generates MR data by performing digital conversion on the MR signals that are output from the reception coil 109. The receiver 110 transmits the generated MR data to the sequence controller 120. The receiver 110 may be to a gantry device that includes the static magnetic field magnet 101 and the gradient coil 103.

The sequence controller 120 images the subject P by driving the gradient power supply 104, the transmitter 108, and the receiver 110 on the basis of sequence information that is transmitted from the calculator 130. The sequence information is information that defines the procedure for performing imaging. The sequence information defines the intensity of the electric current that is supplied by the gradient power supply 104 to the gradient coil 103, the timing at which the electric current is supplied, the intensity of the RF pulse that the transmitter 108 supplies to the transmitter coil 107, the timing at which the RF pulse is applied and the timing at which the receiver 110 detects MR signals. For example, the sequence controller 120 is, for example, an integrated circuit, such as an ASIC (Application Specific Integrated Circuit) or an FPGA (Field Programmable Gate Array), or an electric circuit, such as a CPU (Central Processing Unit) and an MPU (Micro Processing Unit).

Upon receiving the MR data from the receiver 110, which results from the images of the subject P that are imaged by driving the gradient power supply 104, the transmitter 108, and the receiver 110, the sequence controller 120 transfers the MR data received to the calculator 130.

The calculator 130 controls the whole MRI apparatus 100, generates images, etc. The calculator 130 includes an interface unit 131, a storage unit 132, a controller 133, an input unit 134, a display unit 135, and an image generator 136. The controller 133 includes an imaging condition setting unit 133a and a region deriving unit 133b.

The interface unit 131 transmits sequence information to the sequence controller 120 and receives MR data from the sequence controller 120. Upon receiving the MR data, the interface unit 131 stores the received MR data in the storage unit 132. The MR data stored in the storage unit 132 is arrayed into a k-space by the controller 133. As a result, the storage unit 132 stores the k-space data.

The storage unit 132 stores the MR data that is received by the interface unit 131, the k-space data that is arrayed into the k-space by the controller 133, the image data that is generated by the image generator 136, etc. The storage unit 132 is, for example, a RAM (Random Access Memory), a semiconductor memory device such as a flash memory, a hard disk, an optical disk, etc.

The input unit 134 receives various instructions and information inputs from an operator. The input unit 134 includes, for example, a pointing device, such as a mouse or a trackball, and an input device, such as a keyboard. Under the control of the controller 133, the display unit 135 displays various GUIs (Graphical User Interface), images that are generated by the image generator 136, etc. The display unit 135 is, for example, a display device, such as a liquid crystal display.

The controller 133 controls the whole MRI apparatus 100 and controls imaging, image generation, image display, etc. For example, the imaging condition setting unit 133a receives inputs of imaging conditions on the GUI and generates sequence information according to the received imaging conditions. The imaging condition setting unit 133a transmits the generated sequence information to the sequence controller 120. Furthermore, for example, the region deriving unit 133b automatically derives an imaging region and regions relevant to the imaging region (or candidates thereof) by using the imaging conditions that are received by the imaging condition setting unit 133a and images generated by the image generator 136. For example, the controller 133 is an integrated circuit, such as an ASIC or an FPGA, or an electronic circuit such as a CPU or an MPU. The processing performed by the imaging condition setting unit 133a and the region deriving unit 133b will be described in detail below.

The image generator 136 generates an image by reading the k-space data from the storage unit 132, performs reconstruction processing, such as Fourier transformation, on the read k-space data.

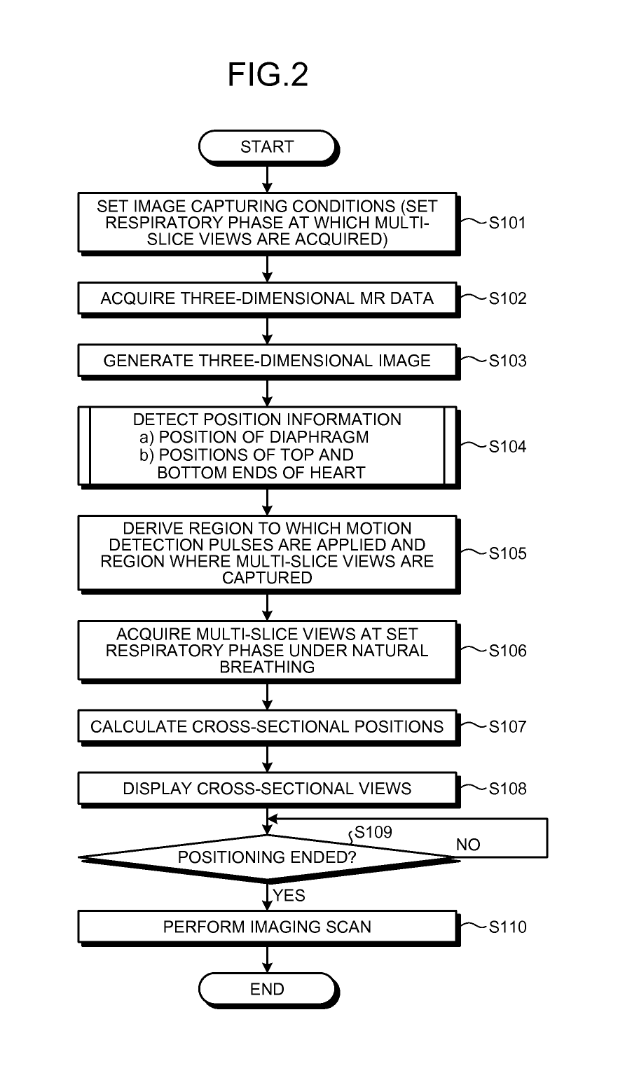

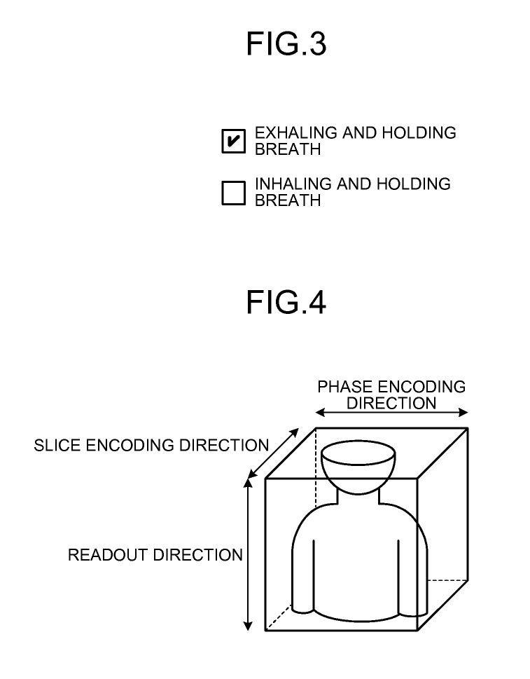

FIG. 2 is a flowchart of a procedure in the first embodiment. As shown in FIG. 2, the MRI apparatus 100 according to the first embodiment can perform the series of processes from setting of imaging conditions to imaging scan of cross-sectional views for diagnosis (hereinafter, “diagnosis cross-sectional views” as required) nearly in an automated flow. Specifically, when a respiratory phase at which multi-slice views are acquired is set in advance and various types of position information are detected from the pre-acquired three-dimensional MR data, the MRI apparatus 100 automatically sets various regions for acquiring multi-slice views on the basis of the detected position information. The regions include a region to which motion detection pulses that are applied for monitoring breathing motions are applied. The MRI apparatus 100 acquires multi-slice views at the pre-set respiratory phase under free breathing, perform positioning of diagnosis cross-sectional views by using the acquired multi-slice views, and performs an imaging scan. The procedure according to the first embodiment will be described with reference to FIGS. 3 to 13.

For “multi-slice view acquisition” in the following embodiment, acquisition in synchronization with the electrocardiogram is supposed. In other words, in the following embodiments, the MRI apparatus 100 performs, for the number of times corresponding to multiple slices, an operation for applying RF pulses (excitation pulses) by using an electrocardiographic signal as a trigger signal and acquiring MR signals corresponding to one slice. In this case, it is desirable that the MRI apparatus 100 finishes acquisition of MR signals corresponding to one slice within one heart rate period (e.g. within 1RR). Acquisition of multiple sets of cross-sectional data performed for multi-slice view acquisition is not limited to one using a 2D sequence, and one using a 3D sequence may be performed. Furthermore, it is desirable that each slice is acquired with the same delay from the trigger signal (e.g. R-wave). “Multi-slice view acquisition” is also referred to as “Multi-slice view imaging”.

At step S101, the imaging condition setting unit 133a receives inputs of imaging conditions from the operator via the input unit 134 on the GUI and, according to the received imaging conditions, generates sequence information. The imaging condition setting unit 133a sets a respiratory phase at which multi-slice views are acquired as one of the imaging conditions.

FIG. 3 is a diagram of a respiratory phase setting GUI in the first embodiment. As shown in FIG. 3, for example, the imaging condition setting unit 133a displays, on the GUI, tick boxes for selecting any one of “exhaling and holding breath” and “inhaling and holding breath” as a respiratory phase and receives the selecting by the operator. The imaging condition setting unit 133a then sets the selected respiratory phase as a respiratory phase at which multi-slice views are acquired. For example, in the first embodiment, the imaging condition setting unit 133a sets “exhaling and holding breath” as the respiratory phase at which multi-slice views are acquired.

“Exhaling and holding breath” represents the respiratory phase of full exhalation by the subject P, in the respiratory cycle during which inhaling and exhaling are repeated. “Inhaling and holding breath” represents the respiratory phase of full inhalation by the subject P. Whether it is desirable that positioning is performed by using multi-slice views at “exhaling and holding breath” or that positioning is performed by using multi-slice views at “inhaling and holding breath” varies depending on the type of the protocol implemented by the subsequent imaging scan. In general, “exhaling and holding breath” is advantageous in that the level of breathing motion tends to be stable. On the other hand, “inhaling and holding breath” is advantageous in that the subject P is load-relieved. For this reason, a proper use is supposed where acquisition is performed at “exhaling and holding breath” for a protocol requiring precision and acquisition is performed at “inhaling and holding breath” for other cases.

At step S102, for which FIG. 2 is referred back, the subject P wearing the receiving coil 109 is placed onto the couchtop 105a of the couch 105 and, when the couchtop 105a is moved to a given position, the sequence controller 120 acquires three-dimensional MR data of a region including the heart and the diaphragm by controlling implementation of pulse sequences according to the sequence information.

FIG. 4 is a diagram for illustrating three-dimensional MR data in the first embodiment. As shown in FIG. 4, for example, the sequence controller 120 acquires three-dimensional MR data at the maximum FOV (Field Of View) that can be set by the MRI apparatus 100 (e.g. the region in which the uniformity of static magnetic field intensity can be secured), with its acquisition center being the center of the magnetic field. As described below, three-dimensional images that are generated from the three-dimensional MR data are used to detect the position of the diaphragm, the position of the top end of the heart, and the position of the bottom end of the heart. For this reason, it is required to acquire three-dimensional MR data of a region including a site that is used as a landmark. For example, in the first embodiment, it is desirable that three-dimensional MR data be acquired, being of a region including heart and the apex of the convex surface of the right diaphragm.

Furthermore, as shown in FIG. 4, the sequence controller 120 sets the head-foot direction for the readout direction, sets the right-left direction for the phase encode direction, sets the dorsoventral direction for the slice encode direction, and acquires three-dimensional MR data. Regarding the information on the position of the diaphragm and information on the position of the heart, the image characteristics on the coronal section are most effective both in automatic detection and in checking of the result of the detection. As for the two directions on the coronal section, that are the head-foot direction and the right-left direction, in case of the right-left direction, there are much less effects of folding outside the imaging region. For this reason, it is desirable that three-dimensional MR data be acquired by using a combination of the above-described encoding directions.

For example, the sequence controller 120 acquires three-dimensional MR data by using a pulse sequence of a GE (Gradient Echo) system. Because the pulse sequence of the GE system is a method of applying excitation pulses of a small flip angle and gradient pulses, the TR (Repetition time) is shorter than that of the pulse sequence of an SE (Spin Echo) system. For example, the sequence controller 120 acquires three-dimensional MR data by using 3D FFE (Fast Field Echo) and 3D SSFP (Steady-State Free Precession). For example, for 3D FFE, various parameters are set on the basis of the time for which breath can be held. For example, the parameters are, without ECG (electrocardiogram), TR/TE (Echo Time)=3.7/1.3 (ms), 92 to 96 (Phase encoding direction)×256 to 366 (readout direction)×30 to 40 (slice encoding direction), etc.

For example, the sequence controller 120 may acquire three-dimensional MR data by performing multi-slice imaging using 2D FFE, 2D SSFP, and 2D FASE. The sequence controller 120 may apply T2 preparation pulses although it involves extension of imaging time. By applying T2 preparation pulses, the image contrast can be enhanced.

In MRI, there is a half-scan method that, without acquiring MR signals for a portion of a region, estimates MR signals of the region where MR signals has not been acquired yet by mathematical processing using complex conjugate properties. For example, a half-scan method corresponding to the phase encoding direction, the slice encoding direction, or both of them may be used together.

At step S103, for which FIG. 2 is referred back, the image generator 136 generates a three-dimensional image by using three-dimensional MR data that is acquired at step S102.

At step S104, the region deriving unit 133b detects the position of the apex of the diaphragm above the lever and the positions of the top end and the bottom end of the heart from the three-dimensional image that is generated at step S103. The position of the apex of the diaphragm is used to derive the region to which motion detection pulses are applied for monitoring the respiratory motion in multi-slice view acquisition. The positions of the top end and bottom end of the heart are used to derive the region where multi-slice views are imaged. For example, the position of the top end of the heart is the position at which the pulmonary artery bifurcates and the position of the bottom end is the position of the apex of the left ventricle.

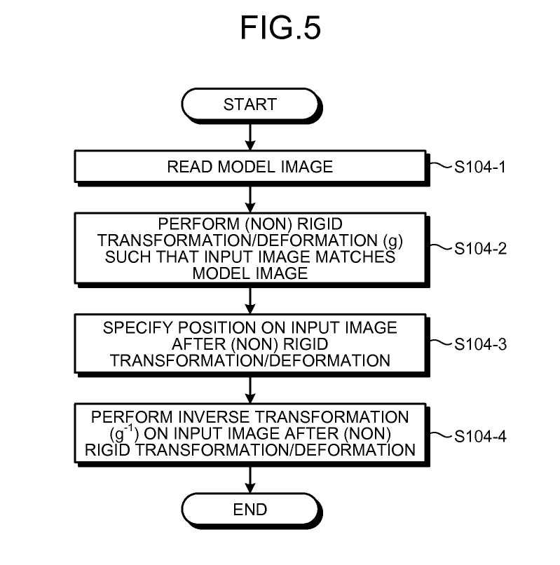

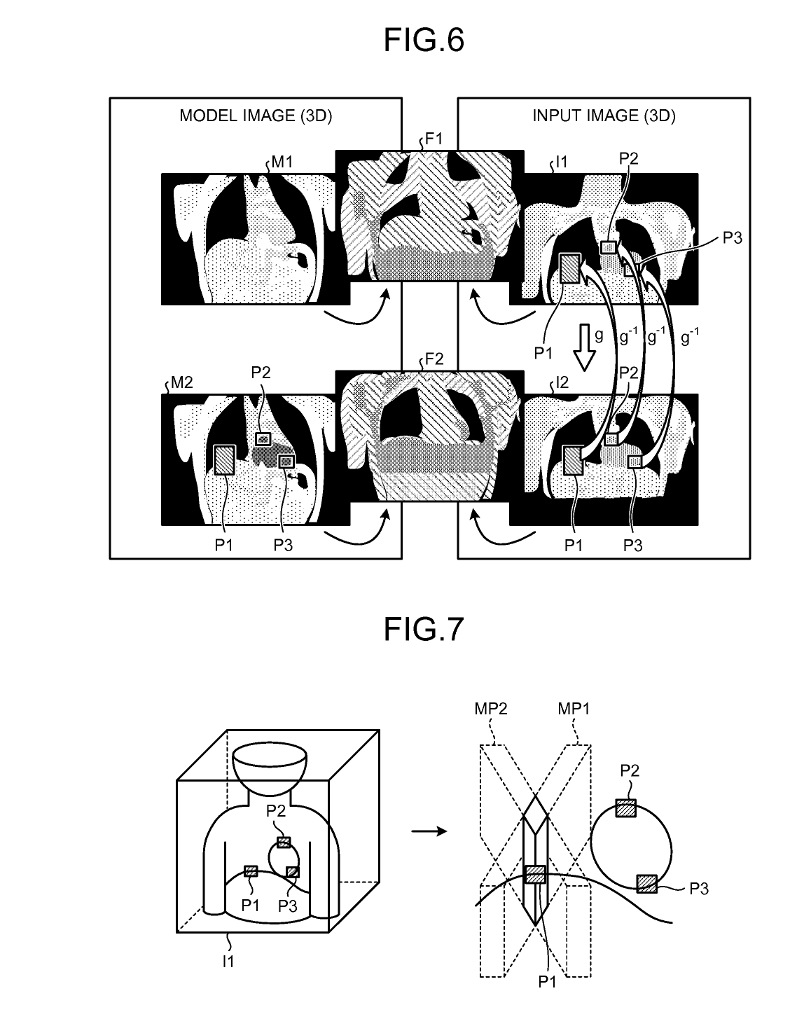

FIG. 5 is a flowchart of a procedure for detecting position information in the first embodiment. FIG. 5 corresponds to the processing at step S104 shown in FIG. 2. FIG. 6 is a diagram for illustrating detection of position information in the first embodiment.

At step S104-1, as shown in FIG. 5, the region deriving unit 133b first reads a model image from the storage unit 132 that stores model images in advance. In this case, the region deriving unit 133b determines imaging conditions (protocol etc.) that are set at step S101 and reads a model image that meets the purposes. In the first embodiment, model images are MR images that are obtained by the MRI apparatus 100 by imaging in advance images of the subject P (e.g., an average patient). Embodiments are not limited to this. For a model image, for example, a mean image from images obtained by imaging images of multiple patients may be used. Alternatively, a model image may be an image that is obtained by performing image processing.

A model image M1 and a model image M2 shown in FIG. 6 are model images where a position P1 of the apex of the convex surface of the right diaphragm, a position P2 of the top end of the heart, and a position P3 of the bottom end of the heart are known and are the same model images. On the other hand, an input image I1 is an image generated at step S103 of FIG. 2 and an input image I2 is an image that is obtained by performing image processing of rigid transformation/deformation or non-rigid transformation/deformation, which will be described below, on the input image I1. A composite image F1 is a composite image of the model image M1 and the input Image I1 and a composite image F2 is a composite image of the model image M2 and the input image I2. Both of the composite image F1 and the composite image F2 are for illustrating the difference between the two images and are not used for position information detection processing performed by the region deriving unit 133b. Both of the images are three-dimensional images.

At step S104-2, for which FIG. 5 is referred back, the region deriving unit 133b performs image processing (g) of rigid or non-rigid transformation/deformation on the input image such that the input image matches the model image. For example, the region deriving unit 133b performs registration in which the image transformation parameters are determined by solving Equation (1):

$$g = {arg min}_{g} ( E ( I ( i ) , M ( g ( i ) ) ) ) \qquad ( 1 )$$

In Equation 1, “i” denotes the position vector of the image, “I(i)” denotes the pixel value of the input image at the position i, and “M(i)” denotes the pixel value of the model image at the position i. The function “E” is an evaluation function of similarity between the input image and the model image. The function “E” is a function of a value that becomes lowered as the similarity increases and is implemented by a sum of squared errors between corresponding pixels, etc. The function “g” is a function of image transformation and is a function of rigid transformation/deformation or non-rigid transformation/deformation, such as affine transformation and thin-plate-spline transformation.

For example, FIG. 6 illustrates that the input image I2 is obtained as a result of performing the image processing (g) for rigid transformation/deformation or non-rigid transformation/deformation on the input image I1 such that the input image matches the model image M1 (or the model image M2). Compared to the composite image F1, the composite image F2 has a smaller difference between the two images.

At step S104-3, for which FIG. 5 is referred back, the region deriving unit 133b specifies the position of the apex of the convex surface of the right diaphragm and the positions of the top end and bottom end of the heart on the input image I2 after the rigid transformation/deformation or non-rigid transformation/deformation. For example, as illustrated in FIG. 6, in the model image M2, the position of the apex of the convex surface of the right diaphragm and the positions of the top end and bottom end of the heart are known three-dimensionally. Accordingly, the position of the apex of the convex surface of the right diaphragm and the positions of the top end and bottom end of the heart are specified also in the same position in the input image I2, which is after the rigid transformation/deformation or non-rigid transformation/deformation performed such that the input image I2 matches the model image M2. Each of the positions may be specified by a point or a region that ranges to some extent.

At step S104-4, the region deriving unit 133b performs image processing (g⁻¹) of inverse transformation on the input image after the rigid transformation/deformation or non-rigid transformation/deformation, into the original input image. Thereafter, as illustrated in FIG. 6, the region deriving unit 133b can specify the position of the apex of the convex surface of the right diaphragm and the positions of the top end and bottom end of the heart on the input image I1 after the inverse transformation.

The method of automatically detecting the position of the apex of the convex surface of the right diaphragm and the positions of the top end and bottom end of the heart are not limited to the above-described registration processing. For example, the region deriving unit 133b may perform automatic detection by performing processing in which surrounding patterns respectively centering the position of the apex of the diaphragm and the positions of the top end and bottom end of the heart are considered and matching is performed by using a mean image as a template or performing processing using a classifier, such as a support vector machine.

At step S105, for which FIG. 2 is referred back, the region deriving unit 133b derives a region to which motion detection pulses are applied and a region where multi-slice views are imaged on the basis of the information on the positions that are detected at step S104.

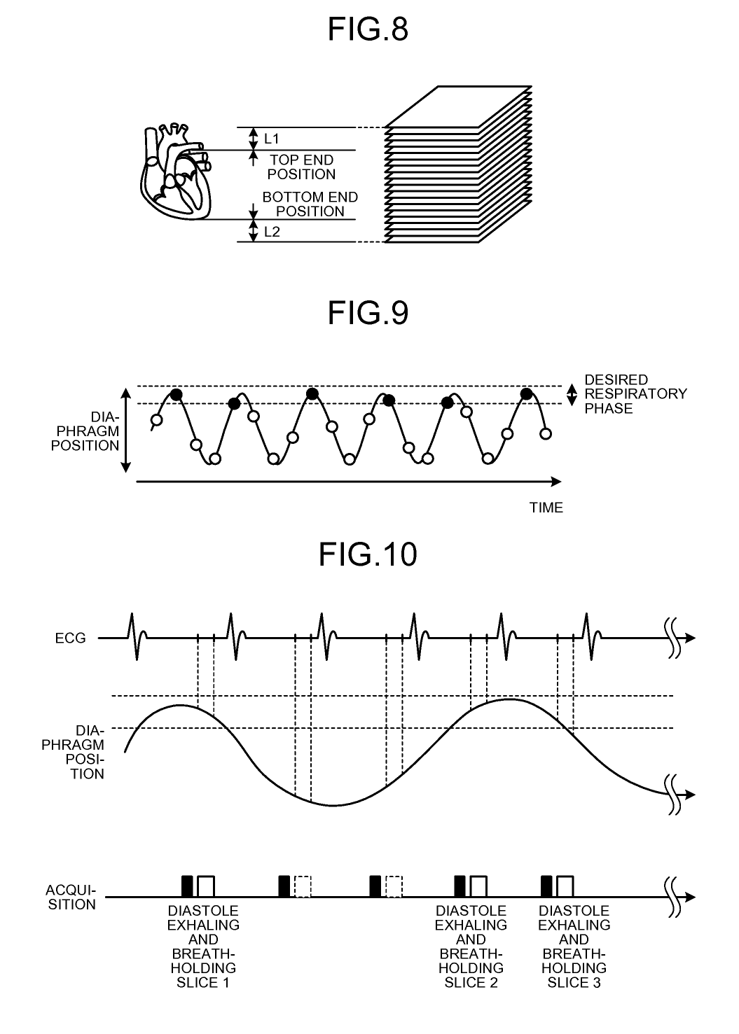

FIGS. 7 and 8 are diagrams illustrating deriving of various regions in the first embodiment. For example, as illustrated in FIG. 7, on the input image I1, the position P1 of the apex of the convex surface of the right diaphragm, the position P2 of the top end of the heart, and the position P3 of the bottom end of the heart are specified. As illustrated in FIG. 7, the size of the cuboids of the regions MP1 and MP2 to which motion detection pulses are applied are predetermined. In the first embodiment, for the system for applying motion detection pulses, is employed a two-plane crossing system in which excitation pulses and re-focus pulses of the SE (Spin Echo) system are caused to cross to excite the region of a form of a quadrangular prism. Thus, the regions to which motion pulses are applied are the two regions MP1 and MP2.

Thus, for example, the region deriving unit 133b sets the cuboid application regions MP1 and MP2 whose sizes are predetermined such that the position P1 of the apex of the convex surface of the right diaphragm is positioned at the center of the intersection of the regions of the form of the quadrangular prisms that cross each other (depicted by the solid line in FIG. 7). The region deriving unit 133b adjusts the extent of the crossing such that the application regions MP1 and MP2 do not overlap the region of the heart that is the target. This is because, in a case where MR data is acquired from the region to which motion detection pulses are applied just before MR data is acquired from the region of the heart, if the application region overlap the region of the heart, an artifact may occur in the image of the heart in relation to the recovery of longitudinal magnetization.

Furthermore, for example, the region deriving unit 133b derives a region where multi-slice views are imaged on the basis of the position P2 of the top end and position P3 of and the bottom end of the heart. As illustrated in FIG. 8, for example, the region deriving unit 133b derives, as the imaging region in the slice direction, a given region including the positions of the top and bottom ends of the heart, i.e., the position at which a given offset L1 is taken from the position of the top end of the heart toward the head direction and the position at which a given offset L2 is taken from the position of the bottom end of the heart toward the foot direction.

Fixed values may be used for the lengths of the offsets L1 and L2 or different variable values may be used per subject P. For example, the region deriving unit 133b may acquire information indicating the body shape in advance, such as the height and weight of the subject P, and information, such as the age, gender, heart rate, pulse rate, history of disease, history of exercise, and history of smoking, of the subject P and may change the lengths of the offsets L1 and L2 according to the information. For example, the region deriving unit 133b may receive, from the operator, a setting about information for which a setting can be made and change the length of the offsets L1 and L2.

It is satisfactory if, for the right-left direction and dorsoventral direction of the region in which multi-slice views are imaged, the region deriving unit 133b uses, for example, predetermined fixed values such that, for example, the region includes at least the heart. Furthermore, for example, the region deriving unit 133b may use different variable values per subject P as in the case of the imaging region in the head-foot direction.

The example has been described above where the sizes of the region to which motion detection pulses are applied and the region where multi-slice views are imaged in the right-left direction and dorsoventral direction are predetermined. However, embodiments are not limited to this. For example, the region deriving unit 133b may adjusts the size and direction of various regions as required on the basis of the information such as the size of the heart or the distance between the apex of the convex surface of the right diaphragm and the heart, specified on the input image. Furthermore, for example, the region deriving unit 133b may set various cuboid regions themselves on the model image. In this case, various regions are considered not to be able to keep their cuboid shapes during the process of inverse transformation. However, the region deriving unit 133b may perform adjustments so that they become cuboid shapes after the inverse transformation.

In this manner, the region deriving unit 133b derives the region to which motion detection pulses are applied and the region in which multi-slice views are imaged. Although it is not described in FIG. 2, the region deriving unit 133b may display, at this stage, a confirmation screen for the operator to confirm various regions that are derived by the region deriving unit 133b.

At step S106, after performing various preparatory scans, the sequence controller 120 acquires multi-slice views at the respiratory phase, which is set at step S101, while the subject P is breathing freely. Furthermore, the sequence controller 120 controls the timings at which MR data of multi-slice views are acquired by detecting breath motions from the position of the apex of the diaphragm and acquires multi-slice views at the desired respiratory phase.

The preparatory scans include scan for acquiring profile data representing the sensitivity in the direction of array of coil elements (or channels), scan for acquiring a sensitivity map representing the distribution of sensitivity of each coil element (or channel), scan for acquiring spectrum data for determining the center frequency of RF pulse, and scan for determining the value of current flowing through the correction coil (not shown) in order to adjust the uniformity of static magnetic field. The preparatory scan is not necessarily performed at this timing. For example, the preparatory scan may be performed after acquisition of multi-slice views. Normally, it is satisfactory if a sensitivity map is acquired before image generation processing.

FIGS. 9 and 10 are diagrams for illustrating acquisition of multi-slice views in the first embodiment. In the first embodiment, the sequence controller 120 detects the position of the apex of the diaphragm by performing one-dimensional Fourier transformation on the MR data that is acquired from the region to which motion detection pulses are applied and then specifies the respiratory phase from the detected apex position. Furthermore, just before acquiring MR data from the region where multi-slice views are imaged, the sequence controller 120 acquires MR data in synchronization with the electrocardiographic signals from the region to which motion detection pulses are applied and, when the specified respiratory phase is a desired respiratory phase, acquires MR data of the multi-slice views.

The open circles and closed circles in FIG. 9 indicate the timings, synchronized with electrocardiographic signals, at which MR data is acquired from the region to which motion detection pulses are applied. When the position of the apex of the diaphragm is within the section indicated by the dotted lines, it is indicated that it is the section of the desired respiratory phase. In other words, the open circles represent acquisition timings synchronized with electrocardiographic signals but do not represent desired respiratory phase. In this case, the sequence controller 120 does not acquire MR data of multi-slice views. On the other hand, the closed circles represent acquisition timings synchronized with electrocardiographic signals and the desired respiratory phase. In this case, the sequence controller 120 acquires MR data of multi-slice views just after motion detection pulses are applied.

FIG. 10 shows electrocardiographic signals, the position of the apex of the diaphragm, and timings at which MR data is acquired. In the first embodiment, multi-slice views at a cardiac phase corresponding to “diastole” and a respiratory phase corresponding to “exhaling and holding breath” are acquired. The sequence controller 120 thus acquires MR data of motion detection pulses at diastolic timings in synchronization with the electrocardiographic signals (the closed rectangles in FIG. 10) and, if the position of the apex of the diaphragm detected from the MR data is within the section of the desired respiratory phase, just thereafter acquires a view corresponding to one slice, being a portion of the multi-slice views (the open rectangles in FIG. 10). On the other hand, when the position of the apex of the diaphragm detected from the MR data of the motion detection pulses is out of the section of the desired respiratory phase, the sequence controller 120 does not acquire multi-slice views just thereafter (the dotted rectangles in FIG. 10).

For example, in the case of the example illustrated in FIG. 10, after a slice 1 from among multi-slice views is acquired, acquisition is not performed at the diastolic timings of twice because it is not within the section of the desired respiratory phase and, thereafter, a slice 2 and a slice 3 are acquired. In this manner, the sequence controller 120 implements a protocol for acquiring multi-slice views for a certain period under free breathing and, for example, multi-slice views corresponding to 18 slices are acquired at the timings of the cardiac phase corresponding to “diastole” and the respiratory phase corresponding to “exhaling and holding breath”. The sequence controller 120 may set a relatively long period in which 18 timings at which the cardiac phase corresponding to “diastole” and the respiratory phase corresponding to “exhaling and holding breath” are secured and implement the protocol. Alternatively, the sequence controller 120 may ends implementation of the protocol at the stage where multi-slice views corresponding to 18 slices are acquired. Here, although “acquisition of multi-slice views” is used for the purpose of explanation, the image generator 136 reconstructs MR data corresponding to one slice that is acquired by the sequence controller 120 so that a view corresponding to 1 slice, being a part of the multi-slice views, are generated.

At step S107, for which FIG. 2 is referred back, the imaging condition setting unit 133a calculates cross-sectional positions serving as information on the positions of the cross-sectional views for diagnosis from the multi-slice views that are acquired by the sequence controller 120. For example, the imaging condition setting unit 133a detects the positions of cardiac characteristic sites from the multi-slice views and, on the basis of the detected positions, calculates all cross-sectional positions of positioning images for positioning the cross-sectional views for diagnosis (e.g. major-axis vector and short-axis vector). Each cross-sectional view that is calculated as a positioning image has a relationship in which the images cross each other. On the basis of the calculated cross-sectional positions, the imaging condition setting unit 133a calculates all cross-sectional views of positioning images.

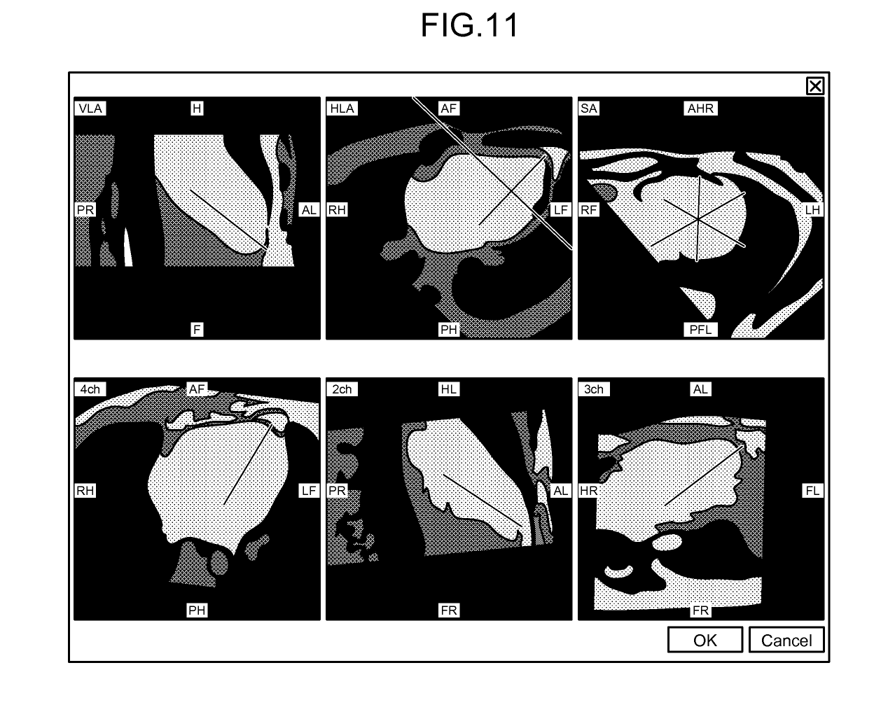

At step S108, the imaging condition setting unit 133a arrays and displays the calculated cross-sectional views, e.g. six cross-sectional views, on the display unit 135. FIG. 11 is a diagram of a positioning GUI in the first embodiment. For example, the imaging condition setting unit 133a arrays and displays, as positioning images, a vertical long axis (VLA) view, a horizontal long axis (HLA) view, a left-ventricle short axis (SA) view, a four-chambers (4ch) cross-sectional view, a two-chambers (2ch) cross-sectional view, and a three-chambers (3ch) cross-sectional view. As illustrated in FIG. 11, the imaging condition setting unit 133a may display information of crossing lines with other cross-sectional views, superimposed onto each cross-sectional view. Although it is not illustrated in FIG. 11 for the purpose of explanation, for example, each of the six kinds of cross-sectional views may be displayed such that they are framed in different colors and such that the colors of the frames are the colors of the information of crossing lines, thereby expressing the crossing lines displayed on each of the cross-sectional views are the crossing lines with which of the cross-sectional views.

At step S109, the imaging condition setting unit 133a receives, from the operator, a positioning operation on the six cross-sectional views that are displayed on the display unit 135 and determines whether the positioning ends.

At step S110, when the positioning ends at step S109, the sequence controller 120 performs imaging scan.

The example has been describe above where six types of basic cross-sectional views are generated as positioning images and imaging scan is performed according to the position of each of the basis cross-sectional views that is determined in the previous processing. However, embodiments are not limited to this. The number and type of cross-sectional views that are generated as positioning images from multi-slice views and whether the cross-sectional views are displayed as a list or individually, etc. can be changed arbitrarily. For example, it is satisfactory if the imaging condition setting unit 133a generates two or more types of cross-sectional views. The cross-sectional views that are generated as positioning images are not limited to cross-sectional views that are defined by the standardization protocol, and the cross-sectional views may be arbitrary cross-sectional views. Furthermore, the number and type of cross-sectional views that are acquired by imaging scan can be changed arbitrarily. For example, it is satisfactory if the sequence controller 120 acquires at least one type of cross-sectional views.

The number and type of cross-sectional views that are generated as positioning images do not necessarily depend on the number and type of cross-sectional views that are acquired by the imaging scan. For example, cross-sectional views that are not scheduled in the initial plan may be acquired due to a subsequent change in the plan.

Positioning of the basis cross-sectional views again each time a new cross-sectional view is acquired would be laborious to the operator. From this viewpoint, if the positioning is finished in advance for more kinds of cross-sectional views than are expected in the imaging scans, it becomes possible to deal with this type of change of plans flexibly.

### Modification 1 of First Embodiment

In the above-described first embodiment, the example has been described where MR data is acquired in synchronization with the electrocardiogram at the timings of one cardiac phase (diastole) in accordance with the timing of respiratory phase. However, embodiments are not limited to this. The sequence controller 120 may acquire MR data at the timings of two or more cardiac phases. In this case, the sequence controller 120 can acquire two sets of multi-slice views at different cardiac phases.

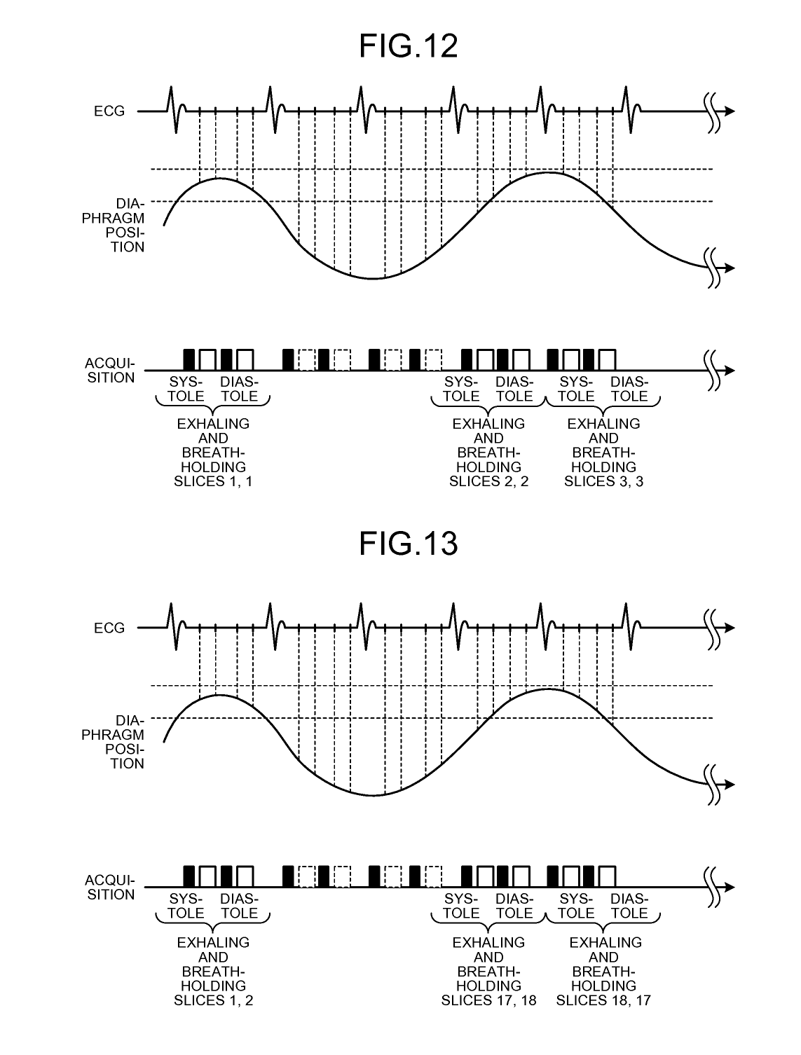

FIG. 12 is a diagram for illustrating acquisition of multi-slice views in the first modification example of the first embodiment. For example, as shown in FIG. 12, the sequence controller 120 acquires MR data of motion detection pulses in synchronization with electrocardiographic signals and, at first, at a systolic timing (the closed rectangles in FIG. 12) and, if the position of the apex of the diaphragm that is detected from the MR data is within the section of the desired respiratory phase, for example, a view corresponding to one slice, being a portion of the multi-slice views, is acquired immediately thereafter (the open rectangles in FIG. 12). On the other hand, if the position of the apex of the diaphragm that is detected from the MR data of motion detection pulses is out of the section of the desired respiratory phase, the sequence controller 120 does not perform acquisition of multi-slice views immediately thereafter (the dotted rectangles in FIG. 12). The sequence controller 120 then acquires MR data of motion detection pulses at the diastolic timing (the closed rectangles in FIG. 12) and, if the position of the apex of the diaphragm that is detected from the MR data is within the section of the desired respiratory phase, for example, a view corresponding to one slice, being a portion of the multi-slice views is acquired immediately thereafter (the open rectangles in FIG. 12). On the other hand, if the position of the apex of the diaphragm that is detected from the MR data of motion detection pulses is out of the section of the desired respiratory phase, the sequence controller 120 does not perform acquisition of multi-slice views immediately thereafter (the dotted rectangles in FIG. 12).

### Modification 2 of First Embodiment

For The first modification example described above, the case has been described where the same slice is acquired at both of the “diastolic” and “systolic” timings. In other words, after a “slice 1” is acquired at the “systolic” timing, a “slice 1” is acquired also at the “diastolic” timing. However, embodiments are not limited to this. FIG. 13 is a diagram for illustrating acquisition of multi-slice views in the second modification example of the first embodiment. For example, as shown in FIG. 13, instead of acquiring the same slices at the “systolic” and “diastolic” timings, the sequence controller 120 acquires one slice in one acquisition and then acquires the next slice in the next acquisition. For example, the sequence controller 120 acquires the “slice 1” at the “systole” and then acquires the “slice 2” at the “diastole” in the same heartbeat. FIG. 13 does not show slices from “slice 3” to “slice 16”. For example, upon ending the acquisition to “slice 18”, the sequence controller 120 performs the acquisition from “slice 18” in the opposite order, thereby acquiring multi-slice views corresponding to 18 slices with respect to each of the “systole” and “diastole”.

### Effects of First Embodiment

As described above, according to the first embodiment, by automatically detecting the position of the apex of the diaphragm from three-dimensional MR data that is acquired in advance and automatically setting a region to which motion detection pulses are applied on the basis of the position of the apex that is automatically detected, acquisition of multi-slice views under free breathing can be implemented easily.

### Second Embodiment

For the first embodiment, the example has been described where MR data is acquired at one respiratory phase. However, embodiments are not limited to this. The sequence controller 120 may acquire MR data at the timings of two or more respiratory phases. In this case, the sequence controller 120 can acquire two sets of multi-slice views at different respiratory phases.

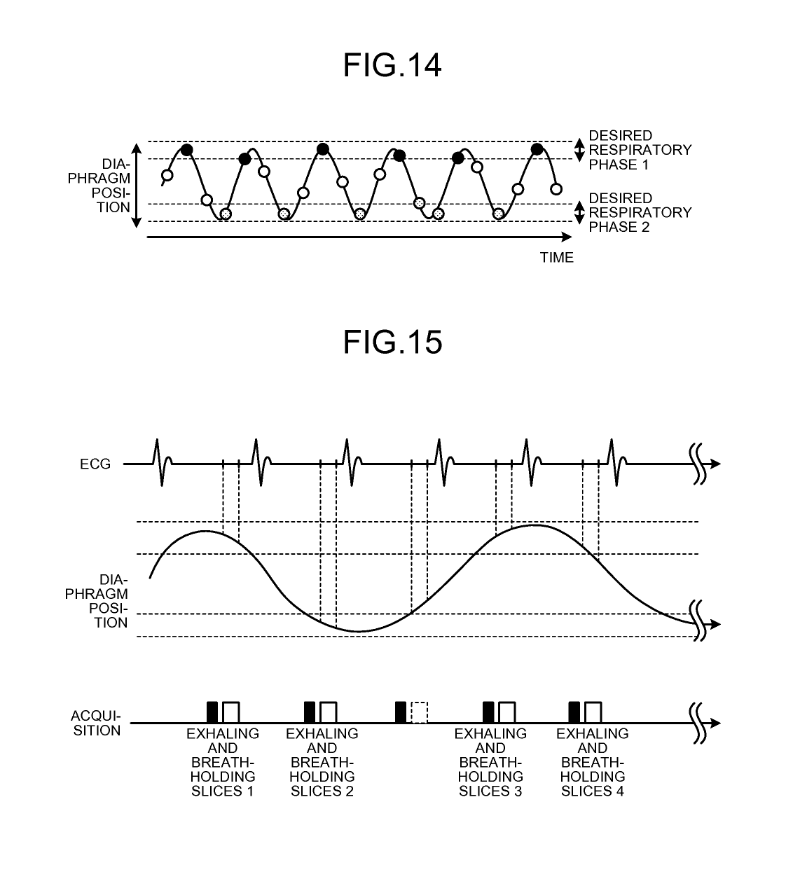

FIGS. 14 and 15 are diagrams for illustrating acquisition of multi-slice views in a second embodiment. As shown in FIG. 14, the desired respiratory phases are the two respiratory phases of the first respiratory phase (exhaling and holding breath) and the second respiratory phase (inhaling and holding breadth).

As illustrated in FIG. 15, in the second embodiment, multi-slice views at the cardiac phase corresponding to “diastole” and the respiratory phase corresponding to “exhaling and holding breath” and multi-slice views at the cardiac phase corresponding to “diastole” and the respiratory phase corresponding to “inhaling and holding breath” are acquired. The sequence controller 120 acquires MR data of motion detection pulses in synchronization with electrocardiographic signals at the diastolic timings (the closed rectangles in FIG. 15) and, if the position of the apex of the diaphragm that is detected from the MR data is any one of the sections of “exhaling and holding breath” and “inhaling and holding breath”, acquires, for example, a view corresponding to one slice, being a portion of the multi-slice views, immediately thereafter (the open rectangles in FIG. 15). On the other hand, if the position of the apex of the diaphragm that is detected from the MR data of the motion detection pulses is not any one of the sections of “exhaling and holding breath” and “inhaling and holding breath”, the sequence controller 120 does not perform acquisition of multi-slice views immediately thereafter (the dotted rectangles in FIG. 15).

In this manner, the sequence controller 120 implements the protocol for acquiring multi-slice views for a certain period under free breathing and acquires, for example, multi-slice views corresponding to 18 slices at each of the timing of a cardiac phase corresponding to “diastole” and a respiratory phase corresponding to “exhaling and holding breath” and the timing of a cardiac phase corresponding to “diastole” and a respiratory phase corresponding to “inhaling and holding breath”. The sequence controller 120 may set a relatively long period in which 18 sets of each of the timings can be secured and implement the protocol. Alternatively, the sequence controller 120 may ends implementation of the protocol at the stage where two sets of multi-slice views corresponding to 18 slices are acquired.

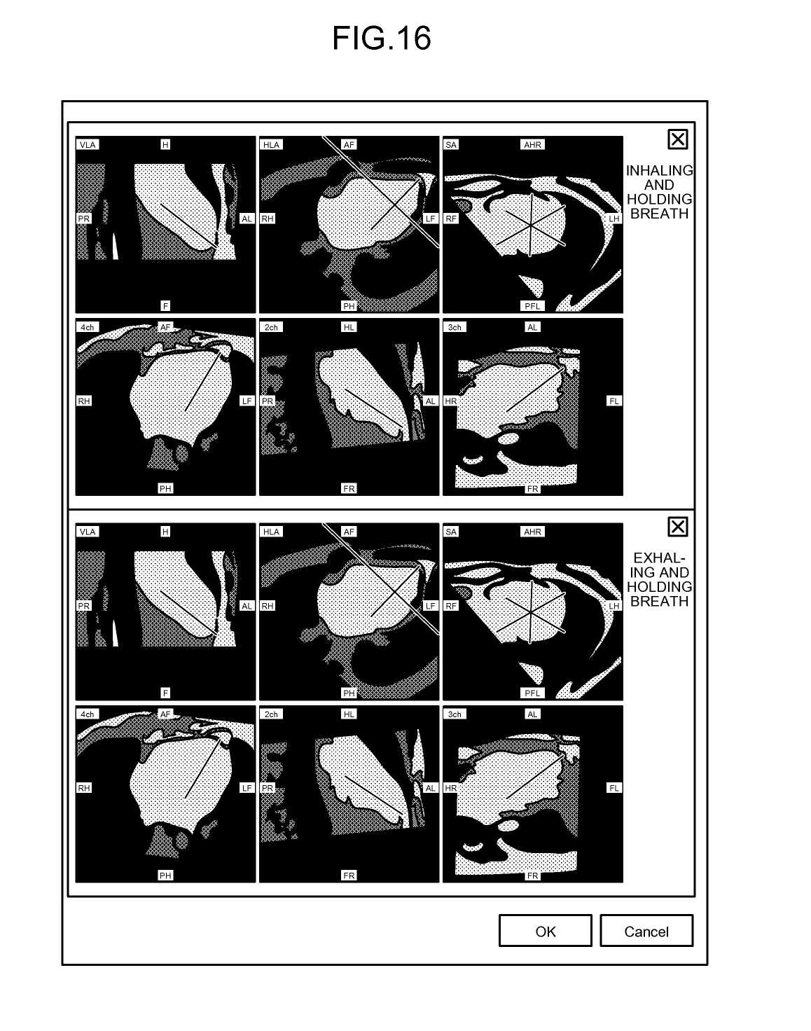

When multiple sets of multi-slice views are acquired as described above, for example, the imaging condition setting unit 133a may array and display the calculated cross-sectional views, e.g. 12 cross-sectional views, on the display unit 135. FIG. 16 is a diagram of a positioning GUI in the second embodiment. As shown in FIG. 16, the imaging condition setting unit 133a arrays and displays, on the display unit 135, six cross-sectional views generated from multi-slice views acquired at the timings of the respiratory phase corresponding to “inhaling and holding breath” and six cross-sectional views generated from multi-slice views acquired at the timings of the respiratory phase corresponding to “exhaling and holding breath”.

### Effects of Second Embodiment

As described above, according to the second embodiment, because two or more respiratory phases are set and multi-slice views corresponding to two or more sets of respiratory phases are acquired simultaneously in pulse sequences executed by the single protocol, multi-slice views corresponding to multiple respiratory phases can be provided to the subsequent processing.

For example, a combination of cross-sectional views for diagnosis and a respiratory phase can be selected appropriately. In the subsequent imaging scan, for example, when imaging scan in which it is preferable to perform positioning using multi-slice views at “inhaling and holding breath” and imaging scan in which it is preferable to perform positioning using multi-slice views at “exhaling and holding breath” are mixed, both of them can be dealt with. Furthermore, if a protocol that is not scheduled initially is added, it can be dealt with without requiring re-acquisition of multi-slice views because multi-slice views corresponding to multiple respiratory phases can be acquired in advance.

Note that the “protocol” is pulse sequence information including information on setting of imaging conditions. The examination performed by using the MRI apparatus 100 includes a group of sequential pulse sequences, such as various types of pre-scan and imaging scan. For each pulse sequence, imaging conditions on the TR (Repetition Time), TE (Echo time), FA (Flip Angle), etc. are set. According to such setting information, the MRI apparatus 100 sequentially implements the group of sequential pulse sequences.

The MRI apparatus 100 manages and provides, as a “protocol”, the pulse sequence information including the information on setting of those imaging conditions (including pre-set information that is set in advance) information. For example, when planning imaging for an examination, an operator, such as a doctor or a technician, calls up a group of protocols that is managed and provided by the MRI apparatus 100 on an imaging planning screen and combines the protocols into the imaging plan while changing the pre-set setting information as required.

The protocol group includes at least one protocol for acquiring a sensitivity map, at least one protocol for shimming, at least one protocol for acquiring multi-slice views, and at least one protocol for imaging. Protocols for imaging are aimed differently, i.e., there are a protocol for acquiring a basic cardiac cross-sectional view, a protocol for imaging the running of the coronary artery over the heart, and protocol for acquiring cine-images. In other words, one “protocol” can be referred to as a unit of pulse sequences that are implemented consecutively as a series of processes without any wait time due to, for example, some operation by the operator.

### Other Embodiments

Embodiments are not limited to the above-described first and second embodiments.

Breath Phase Setting GUI

In the above-described embodiment, as a respiratory phase setting GUI, the GUI has been described that displays tick boxes for selecting any one of “exhaling and holding breath” and “inhaling and holding breath” as a respiratory phase. However, embodiments are not limited to this.

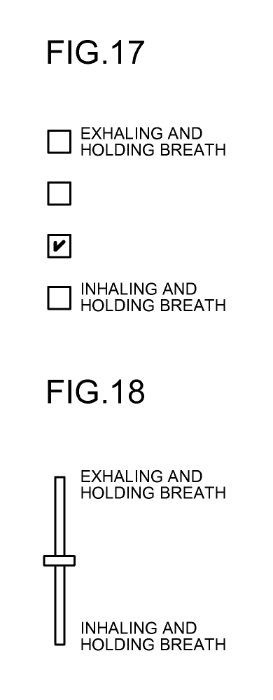

FIGS. 17 and 18 are diagrams of respiratory phase setting GUIs in one of the other embodiments. As shown in FIG. 17, the imaging condition setting unit 133a may display, as a GUI, tick boxes with which a middle respiratory phase between “exhaling and holding breath” and “inhaling and holding breath” can be selected, as the respiratory phase. Alternatively, for example, a slider-type GUI may be displayed as shown in FIG. 18. In this case, the operator can set an arbitrary respiratory phase by adjusting the adjuster via the input unit 134, such as a mouse.

Non-Selective Acquisition

For the above-described embodiments, the example has been described where data at a desired cardiac phase from among cardiac phases is selectively acquired and the example where data at a desired respiratory phase among respiratory phases is selectively acquired. However, embodiments are not limited to this. For example, the sequence controller 120 may acquire MR data of multi-slice views consecutively, independent from the cardiac cycle and breath cycle of the subject. In this case, concurrently with this, the MRI apparatus 100 acquires data of electrocardiographic signals and breath signals together. By using the data of electrocardiographic signals and breath signals, the image generator 136 specifies MR data corresponding to a desired cardiac phase and respiratory phase from among the consecutively acquired MR data of multi-slice views, and, by using the specified MR data, the image generator 136 selectively generates multi-slice views at the desired cardiac phase and respiratory phase.

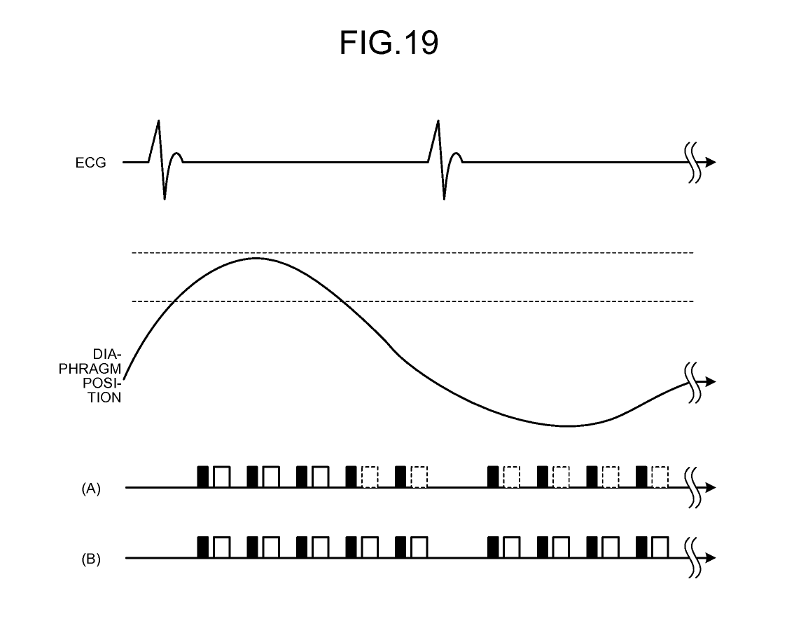

FIG. 19 is a diagram for illustrating acquisition of multi-slice views in one of the other embodiments. As shown in FIG. 19(A), for example, the sequence controller 120 acquires MR data at timings of the respiratory phase corresponding to “inhaling and holding breath” and does not acquire MR data in other timings but, within the section of the respiratory phase corresponding to “inhaling and holding breath”, acquires MR data consecutively, independent from the cardiac cycle. Alternatively, as shown in FIG. 19(B), for example, the sequence controller 120 consecutively acquires MR data, independent from the cardiac cycle and respiratory cycle.

Region to which Motion Detection Pulses are Applied

In the above-described embodiments, a region to which motion detection pulses are applied is determined by using the apex of the convex surface of the right diaphragm as a landmark. However, embodiments are not limited to this. For example, the position of the apex of the diaphragm above the spleen (the left diaphragm (the ventricular apex)) may be detected as a landmark to determine a region to which motion detection pulses are applied. In this case, for example, the region deriving unit 133b may determine multiple candidates of the application region, display the application region on a confirmation screen, and receives selecting by the operator. Alternatively, for example, the region deriving unit 133b may determine a more appropriate application region and display only the most appropriate application region on a confirmation screen or display application regions with the order of priority. This determination can be made, for example, according to overlapping with the region where cardiac images are imaged. The content of the above descriptions, including the determination of multiple candidates, can be also applied to other embodiments.

Furthermore, for example, in the above-described embodiments, the two-plane crossing system is described as the system for applying motion detection pulses. However, embodiments are not limited to this. For example, a pencil-beam system that is used in a pulse sequence of a GE system may be used.

Furthermore, for example, for the above-described embodiments, the method using a “1D Motion Probe” for detecting the amount of shift of the diaphragm by performing one-dimensional Fourier transformation on MR data that is acquired from the region to which motion detection pulses are applied is described. However, embodiments are not limited to this. For example, a method using a “2D Motion Probe” may be employed. In “2D Motion Probe”, two-dimensional Fourier transformation is performed on MR data that is acquired from a region to which motion detection pulses are applied and, on the basis of the imaged data, for example, the amounts of shift of the diaphragm in the vertical direction and anteroposterior direction are detected. In this case, the cross-sectional plane setting of the “2D Motion Probe” can be set, for example, as the 2D horizontal cross-sectional plane whose axis is the line along the body axis direction, the line passing through the specified position (point) of the apex of the diaphragm. Alternatively, since the positions of important internal organs or the vascular system can be specified, it is possible to perform the cross-sectional plane setting in an angle that avoids these important organs, with the line along the body axis passing through the position (point) of the apex of the diaphragm being set as the axis.

Target Internal Organ

In the first embodiment, the heart is considered as a target internal organ. However, embodiments are not limited to this and other internal organs may be considered. For example, the lever may be a target.

Setting of Breath Phase

For the above-described embodiments, the method of receiving, by a setting by the operator, a respiratory phase at which multi-slice views are acquired is described. However, the embodiments are not limited to this. For example, when imaging conditions are set, the imaging condition setting unit 133a may display an imaging condition setting GUI and receive a designation of a protocol from the operator. For example, according to the received designation of a protocol for imaging scan, the MRI apparatus 100 may determine a desired respiratory phase and the result of the determination may be reflected to control on the timing implemented by the sequence controller 120 and control implemented when the image generator 136 selectively generates images later.

For example, when the protocol that is selected as a protocol implemented in imaging scan is a protocol suitable for a respiratory phase corresponding to “inhaling and holding breath”, the sequence controller 120 controls timings such that multi-slice views are acquired at the timing of the respiratory phase corresponding to “inhaling and holding breath”. Furthermore, for example, when the protocol that is selected as a protocol implemented in imaging scan is a protocol suitable for both a respiratory phase corresponding to “inhaling and holding breath” and a respiratory phase corresponding to “exhaling and holding breath”, the sequence controller 120 controls timings such that multi-slice views are acquired at the timings of the respiratory phase corresponding to “inhaling and holding breath” and the respiratory phase corresponding to “exhaling and holding breath”.

Deriving of Other Regions

For the above-described embodiments, the example has been described where, in addition to an imaging region, a region to which motion detection pulses are applied is derived from MR data that is acquired to derive regions. However, embodiments are not limited this. The region deriving unit 133b can derive, from the MR data acquired for deriving regions, regions to which various pulses are applied, which involves setting of spatial positions. For example, the region deriving unit 133b can derive a region (at least one region) to which saturation pulses or other ASL pulses are applied.

The region deriving unit 133b may not only derive regions to which various pulses are applied from MR data that are acquired for deriving regions, but also derive other regions. For example, the region deriving unit 133b may detect a cuboid region that makes external contact with the subject P from the MR data and derive a region wider than the cuboid region as an imaging region where a sensitivity map is captured. Alternatively, for example, the region deriving unit 133b may detect a cuboid region that makes external contact with the heart from MR data and derive a given region including the cuboid region as an imaging region where images are imaged by shimming.

Image Processing

Image processing for deriving regions is not limited to this. For the above-described embodiments, the method has been described where registration is performed such that an input image matches a model image. However, embodiments are not limited to this. For example, a method may be used in which a model image is transformed and registration between the transformed model image and an input image is performed to derive each region. Alternatively, for example, the region deriving unit 133b may derive an imaging and regions relevant to the imaging region by using a method using no model image. For example, the region deriving unit 133b performs threshold processing on a three-dimensional image to perform segmentation between the regions of the air and the regions of other than the air. By applying a diaphragm surface model and a spherical model imitating the heart to the boundary of the region of the air, the region deriving unit 133b detects the heart and the position of the apex of the convex surface of the diaphragm. The region deriving unit 133b uses the position as a landmark to derive a region where cardiac images are imaged and a region to which motion detection pulses are applied.

For the above-described embodiment, the image processing using a model image has been described. However, multiple types of model images may be prepared according to, for example, the age and anamnesis. In the above-described embodiments, the method has been described where a model image is selected according to the imaging conditions that are input. However, for example, the region deriving unit 133b may select an appropriate model image according to information that is input as items for the examination, such as the age and anamnesis of the subject P.

For the above-described embodiments, the method for selecting a model image according to the imaging conditions that are input is described. However, embodiments are not limited to this. For example, let us assume that MR data is acquired for deriving regions and three-dimensional images that are generated from the MR data are stored in a data structure according to the DICOM (Digital Imaging and Communications in Medicine) standards in the storage unit 132. In this case, the region deriving unit 133b may, for example, select a model image etc. according to associated information that is associated with the three-dimensional image (e.g. “Heart”, “3D FFE” etc.). The associated information is not limited to associated information according to the DICOM standards. The associated information may be associated information uniquely associated with the MRI apparatus 100.

Multi-Slice Views at Given Breath Phase Acquired for Positioning

For the above-described embodiments, has been described the method in which three-dimensional MR data is acquired prior to acquisition of multi-slice views and, on the basis of the information on the position detected from the three-dimensional MR data, various regions (e.g. a region to which motion detection pulses are applied) for acquiring multi-slice views are automatically set. However, embodiments are not limited to this. Acquisition of three-dimensional MR data is not an essential element and automatic setting of various regions for acquiring multi-slice views is not an essential element.

In the above-described embodiments, breath motions are monitored by using the method in which motion detection pulses are applied. However, embodiments are not limited to this. For example, respiratory motions may be monitored by using a method implemented with a respiratory sensor that the subject P wears. For example, the respiratory sensor detects motions resulting from breathing as an air pressure and converts the detected air pressure into electronic signals and outputs the electronic signals as respiratory signals.

In other words, the MRI apparatus 100 acquires data of multi-slice views at a given respiratory phase by performing imaging in synchronization with breathing using some method and, from the acquired data of multi-slice views, calculates cross-sectional position information that is information on the position of cross-sectional views that are acquired by imaging scan. On the basis of the calculated cross-sectional position information, the MRI apparatus 100 performs imaging scan.

Specific Values and Order of Processing

In principle, the specific values and order of processing illustrated for the above-described embodiments are mere examples. For example, the landmarks used to derive various regions may be changed arbitrarily. Furthermore, the order of processing, such as the order of processing not displaying a confirmation screen, may be changed arbitrarily. For example, the example has been described where, in the procedure shown in FIG. 2, a respiratory phase is set at step S101, but embodiments are not limited to this. It is satisfactory if a respiratory phase is set until the timing (step S106) at which multi-slice views are acquired. Specific pulse-sequences may be changed arbitrarily.

In the above-described embodiment, “systole” and “diastole” are exemplified as cardiac phases and “inhaling and holding breath” and “exhaling and holding breath” are exemplified as respiratory phases, and given combinations thereof are exemplified and described. However, they are mere examples. An arbitrary change, such as a combination other than that of the above-described embodiments or a combination of cardiac phase or respiratory phase other than those exemplified in the above-described embodiments, may be made.

Control on Breath Phase in Imaging Scan

For the above-described second embodiment, the example has been described where multi-slice views are acquired at two or more respiratory phases. In this case, as described above, multi-slice views corresponding to multiple respiratory phases can be provided to the subsequent imaging scan. For example, when imaging scan is performed, the respiratory phase at which image data is acquired can be switched appropriately. An embodiment relevant to control on respiratory phases in imaging scan will be described below regarding a case where multi-slice views are acquired at two or more respiratory phases.

For example, if imaging scan is for acquiring image data for diagnosis at least any one of “exhaling and holding breath” and “inhaling and holding breath”, the sequence controller 120 appropriately switches, when performing imaging scan, the information on positioning that is used when image data is acquired.

Specifically, when acquiring image data at “exhaling and holding breath”, the sequence controller 120 acquires image data on the basis of the information on the positioning that is performed by using multi-slice views that are acquired at “exhaling and holding breath” and, when acquiring image data at “inhaling and holding breath”, the sequence controller 120 acquires image data on the basis of the information on the positioning that is performed by using multi-slice views that are acquired at “inhaling and holding breath”.

In this case, for example, the imaging condition setting unit 133a sets any one of “exhaling and holding breath” and “inhaling and holding breath” as a respiratory phase at which image data is acquired in imaging scan. When performing imaging scan, the sequence controller 120 acquires image data at the respiratory phase that is set by the imaging condition setting unit 133a.

For example, the imaging condition setting unit 133a receives an operation for selecting any one respiratory phase of “exhaling and holding breath” and “inhaling and holding breath” from the operator and sets the selected respiratory phase selected by the operation as a respiratory phase at which image data for diagnosis is acquired in imaging scan.

In imaging scan, multiple protocols can be implemented. For example, in a cardiac examination method using an MRI apparatus, because multiple types of examinations are carried out, pre-determined multiple protocols determined per examination are sequentially implemented as the imaging scans. For example, in a cardiac examination method using an MRI apparatus, a cine examination, a flow examination, a perfusion examination, an LGE (Late Gadolinium Enhancement) examination, and a coronary artery examination are performed.

The cine examination is an examination for observing the shape and motions of the cardiac muscle and valves, where a protocol for acquiring cine images is implemented. The flow examination is an examination for determining whether there is a backward flow of the blood, where a protocol for imaging the speed of the blood flow is implemented. The perfusion examination is an examination for determining whether there is ischemia, where a protocol for acquiring perfusion images using a contrast agent is implemented. The LGE examination is an examination for determining whether there is myocardial infarction, where a protocol for acquiring delay contrast images is implemented. The coronary artery examination is an examination for determining whether there is a stricture in the coronary artery, where a protocol for imaging the running of the coronary artery over the whole heart is implemented.

Among these protocols, the protocols of the cine examination, the flow examination, the perfusion examination, and the LGE examination, image data is acquired in a state where the subject is holding breath. According to these protocols, on the basis of the information on positioning that is performed by using multi-slice views that are acquired in advance, image data is acquired. According to the protocol of the coronary artery examination, the data of images over the whole heart is acquired under free breathing.

For example, according to the protocol that is used for the cine examination and flow examination, breath-holding for 10 to 20 seconds is performed repeatedly for about 10 to 20 times. According to the protocol used for the perfusion examination, in order to observe the state of perfusion of the contrast agent over the whole heart, breath-holding is performed for about one minute. According to the protocol used for the LGE examination, in order to observe a part where the contrast agent cannot flow completely, breath-holding for about 20 seconds is repeated for about five times.

As described above, when the imaging scan is for sequentially implementing multiple protocols, for example, the imaging condition setting unit 133a sets, for each of the protocols for acquiring image data in a state where the subject is holding breath, a respiratory phase at which image data is acquired. When imaging scan is performed, the sequence controller 120 acquires, for each of the protocols, on the basis of the information on the positioning performed by using the multi-slice views that are acquired at the respiratory phase that are set by the imaging condition setting unit 133a, image data for diagnosis at the respiratory phase.

For example, before execution of imaging scan is started, for each of the protocols for acquiring image data in a state where the subject is holding breath, the imaging condition setting unit 133a receives a designation of a respiratory phase from the operator via the same GUI as that shown in FIG. 3, FIG. 17, or FIG. 18. For each of the protocols, the imaging condition setting unit 133a sets the respiratory phase received from the operator as the respiratory phase at which image data is acquired.

For example, the imaging condition setting unit 133a may set a respiratory phase of all protocols before implementation of the first protocol is started or set a respiratory phase of the protocol to be implemented next just before each protocol is started. Alternatively, in response to a request from the operator and at an arbitrary timing, the imaging condition setting unit 133a may set a respiratory phase of a protocol that is specified by the operator.

In this manner, by setting a respiratory phase at which image data is acquired for each of the protocols for acquiring image data in a state where the subject is holding breath, for example, the respiratory phase can be switched per protocol according to the state of the examination and the condition of the patient who is the subject.

It is generally known that, compared to “inhaling and holding breath”, at “exhaling and holding breath”, the position of the diaphragm when breath is held is stable but the load on the patient that is the subject is large. On the other hand, it is known that, compared to “exhaling and holding breath”, at “inhaling and holding breath”, the position of the diaphragm when breath is held is unstable but the load on the patient that is the subject is small.

For this reason, respiratory phases of each of the protocols are set, so that, in the case of a cine examination, where, for example, in a heart examination, the breath-holding time is relatively short and, since multiple cross-sectional positions are imaged, divided into several times, higher level of precision of the respiratory position is required, imaging data is acquired at “exhaling and holding breath”, in which the position of the diaphragm is stable, and in the case of other protocols, respiratory phases of each of the protocols are set, so that, imaging data is acquired at “inhaling and holding breath”, in which the load to the patient is smaller. Thus, according to the precision required for the examination and the load on the patient, the respiratory phase at which image data is acquired can be switched appropriately.

By setting a respiratory phase just before each protocols is started, for example, in a case where the protocol for acquiring image data at “exhaling and holding breath” is continued, if the fatigue of the patient becomes larger than expected during the examination process, the respiratory phase can be switched for the subsequent protocols such that image data is acquired at “inhaling and holding breath” at which the load is small. Accordingly, the load on the patient who is the subject due to breath-holding can be reduced and the situation where the examination has to be discontinued due to the fatigue of the patient can be avoided.

Methods of setting a respiratory phase at which image data is acquired in imaging scan are not limited to the above method.

For example, when a protocol for acquiring image data in a state where the subject is holding breath is implemented, the subject tends to be notified of, at the timing when the protocol is implemented, a respiratory phase at which the patient holds the breath. For example, the sequence controller 120 gives a notification representing the respiratory phase at which the subject holds the breath by voice via an audio microphone that is provided to the MRI apparatus 100. For example, when implementing a protocol for acquiring image data in a state where the subject is holding breath at any one of “exhaling and holding breath” and “inhaling and holding breath”, the sequence controller 120 makes a notification representing any one of “exhaling and holding breath” and “inhaling and holding breath” as a respiratory phase at which the patient holds the breath.

In such a case, for example, before imaging scan is performed, the operator selects which of “exhaling and holding breath” and “inhaling and holding breath” is given to the subject as a notification representing the respiratory phase at which the subject holds the breath in the imaging scan. For example, the imaging condition setting unit 133a may set a respiratory phase at which image data is acquired in imaging scan in synchronization with the selecting of a respiratory phase by the operator.

For example, the imaging condition setting unit 133a receives, from the operator, an operation for selecting which of “exhaling and holding breath” and “inhaling and holding breath” is given to the subject as a notification representing a respiratory phase at which the subject holds the breath in imaging scan. The imaging condition setting unit 133a also sets the respiratory phase selected by the operation as a respiratory phase at which image data for diagnosis is acquired in imaging scan.

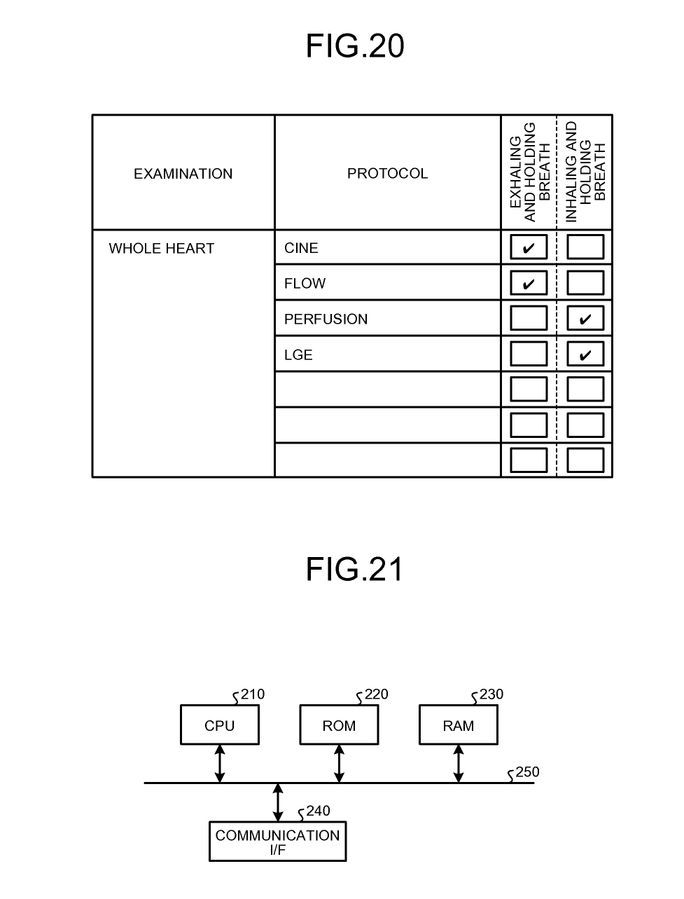

FIG. 20 is a diagram of a respiratory phase setting GUI in one of the other embodiments. For example, as shown in FIG. 20, the imaging condition setting unit 133a displays, on the display unit 135, a GUI in a form of a list in which two tick boxes corresponding to “exhaling and holding breath” and “inhaling and holding breath”, respectively, are arrayed for each of multiple protocols.

The example shown in FIG. 20 represents exemplary protocols implemented in a cardiac examination. i.e., “Whole Heart”, and “Cine” represents a protocol for a cine examination, “Flow” represents a protocol for a flow examination, “Perfusion” represents a protocol for a perfusion examination, and “LGE” represents a protocol for an LGE examination.

For example, before the first protocol in imaging scan is implemented, the imaging condition setting unit 133a displays the GUI shown in FIG. 20 on the display unit 135 in response to a request from the operator. The imaging condition setting unit 133a then receives, from the operator, an operation for ticking any one of the tick boxes of “exhaling and holding breath” and “inhaling and holding breath” for each protocol via the displayed GUI. The imaging condition setting unit 133a then sets the respiratory phase of the ticked box as a respiratory phase at which image data is acquired in imaging scan.

As described above, the imaging condition setting unit 133a can set a respiratory phase in imaging scan efficiently by setting a respiratory phase at which image data is acquired in imaging scan in synchronization with an operation for selecting a respiratory phase that is given to the subject as a notification representing a respiratory phase at which the subject holds the breath.

The example has been described where specifying of a respiratory phase is received from the operator. However, methods of setting a respiratory phase are not limited to this. For example, a respiratory phase at which image data is acquired may be set on the basis of the protocol that is specified by the operator when imaging is planned, information on the patient that is acquired from another system, etc.

For example, the imaging condition setting unit 133a receives, from the operator, an operation for specifying a protocol that is implemented in imaging scan and sets a respiratory phase at which image data is acquired according to the protocol that is specified by the operation.

For example, as described above, in a case where the MRI apparatus 100 manages and provides a protocol group of multiple protocols per unit of examination, a respiratory phase at which image data is acquired is included in advance in the information on setting of each protocol used in imaging scan. The information representing the respiratory phase is, for example, information representing “exhaling and holding breath” and information representing “inhaling and holding breath”.

The imaging condition setting unit 133a receives, from an operator, such as a doctor or a technician, an operation for selecting a desired protocol group including a protocol that is implemented in imaging scan from among protocol groups that are provided when the operator plans imaging. The operator appropriately selects, from among the protocol groups that are managed and provided by the MRI apparatus 100, a protocol group according to the site to be examined and the type and purpose of the examination.

The imaging condition setting unit 133a receives, from the operator, an operation for specifying at least one protocol by receiving an operation for adding a necessary protocol or deleting an unnecessary protocol with respect to the selected protocol group. For example, the imaging condition setting unit 133a reads information on setting of the specified protocols from among information on setting of protocols that is stored in advance in the storage unit 132. On the basis of the information representing the respiratory phase contained in the read setting information, the imaging condition setting unit 133a sets a respiratory phase at which image data is acquired in the protocol implemented in the imaging scan.

In this manner, by automatically setting a respiratory phase in imaging scan on the basis of the information representing the respiratory phase contained in the information of the protocol group that is managed and provided by the MRI apparatus 100, the load on the operator in setting a respiratory phase can be reduced.

For example, the MRI apparatus 100 manages and provides the protocol groups separately according to the purposes of examinations, for the same type of examinations, such as, for youth and seniors and for new patients and followed-up patients. For example, in such a case, for each protocol group, a respiratory phase at which image data is acquired may be changed even between the same type of protocols.

For example, regarding a cardiac examination, for all protocols of a protocol group for youth and new patients, a respiratory phase of each protocol is set such that image data is acquired at “exhaling and holding breath” at which the position of the diaphragm is stable. For example, similarly, regarding a cardiac examination, for a protocol group for seniors and followed-up patients, a respiratory phase of each protocol is set such that, in a cine examination that requires high precision, image data is acquired at “exhaling and holding breath” at which the position of the diaphragm is stable and, for other protocols, image data is acquired at “inhaling and holding breath” at which the load on the patient is small. Accordingly, according to the purpose of the examination, the respiratory phase at which image data is acquired can be switched appropriately.

The imaging condition setting unit 133a may display the GUI shown in FIG. 20 on the display unit 135 in response to a request from the operator and, for each protocol, information representing the respiratory phase that is set according to the protocol information may be displayed in a tick box. The imaging condition setting unit 133a may receive, per protocol, an operation for changing the respiratory phase via the GUI and change the respiratory phase that is already set. Accordingly, the operator may appropriately change the automatically-set respiratory phase at an arbitrary timing according to the state of the examination and the condition of the subject.

For example, the imaging condition setting unit 133a may acquire attribute information on a subject to be examined or information on a past examination and, on the basis of the acquired information, set a respiratory phase at which image data is acquired in imaging scan.

For example, when the MRI apparatus 100 is connected to another system that manages information on a patient who is a subject via a network, the imaging condition setting unit 133a acquires attribute information on the subject to be examined or information on the past examination from the system. The other system is, for example, a hospital information system (HIS) or a radiology information system (RIS).

For example, according to the acquired attribute information on the subject, the imaging condition setting unit 133a sets a respiratory phase at which image data is acquired in imaging scan. For example, when the subject is at a given age or younger, for all of multiple protocols implemented in the cardiac examination, the imaging condition setting unit 133a sets a respiratory phase such that image data is acquired at “exhaling and holding breath” at which the position of the diaphragm is stable. On the other hand, when the subject is older than the given age, for the cine examination that requires high accuracy, the imaging condition setting unit 133a sets a respiratory phase such that image data is acquired at “exhaling and holding breath” at which the position of the diaphragm is stable and, for other protocols, image data is acquired at “inhaling and holding breath” at which the load on the subject is small. Accordingly, according to the attribute of the subject, the respiratory phase at which image data is imaged in imaging scan can be switched appropriately.

For example, on the basis of the acquired information on the past examination of the subject, the imaging condition setting unit 133a sets a respiratory phase at which image data is acquired in imaging scan. For example, when the patient is new, regarding all of the multiple protocols implemented in the cardiac examination, the imaging condition setting unit 133a sets a respiratory phase such that image data is acquired at “exhaling and holding breath” at which the position of the diaphragm is stable. On the other hand, when the subject had the same cardiac examination before and there is an examination that should be particularly weighted from among the multiple examinations included in the cardiac examination, respiratory phases are set such that image data is acquired for the examination at “exhaling and holding breath” at which the position of the diaphragm is stable and, for other protocols, image data is acquired at “inhaling and holding breath” at which the load on the patient is small. Thus, according to the state of the examination of the subject, the respiratory phase at which image data is acquired in imaging scan can be switched appropriately.

### Setting of Cardiac Phase

For the above-described embodiments, the case has been described where, when multi-slice views are acquired in synchronization with the electrocardiogram, for example, multi-slice views are acquired at a given cardiac phase, i.e., at any one of or both of “exhaling and holding breath” and “inhaling and holding breath”. The setting of the given cardiac phase is, for example, performed according to a protocol that is specified by the operator when planning imaging or information on the subject that is acquired from another system.

For example, the imaging condition setting unit 133a receives, from the operator, an operation for specifying a protocol for acquiring multi-slice views and, according to the protocol specified by the operation, sets a cardiac phase at which multi-slice views are acquired.

For example, as described above, in a case where the MRI apparatus 100 manages and provides a protocol group of multiple protocols per unit of examination, information representing a cardiac phase at which multi-slice views are acquired is included in advance in the information on setting of protocols for acquiring multi-slice views. For example, the information representing the cardiac phase is, for example, information representing “diastole” and information representing “systole”.

The imaging condition setting unit 133a receives, from an operator, such as a doctor or a technician, an operation for selecting a desired protocol group including a protocol for acquiring multi-slice views from among protocol groups that are provided when the operator plans imaging. The operator appropriately selects, from among the protocol groups that are managed and provided by the MRI apparatus 100, a protocol group according to the site to be examined and the type and purpose of the examination.

For example, the imaging condition setting unit 133a reads information on setting of a protocol for acquiring multi-slice views contained in the selected protocol group from among information on setting of protocols stored in advance in the storage unit 132. On the basis of the information representing the cardiac phase contained in the read setting information, the imaging condition setting unit 133a sets the cardiac phase at which multi-slice views are acquired.

In this manner, by automatically setting a cardiac phase at which multi-slice views are acquired on the basis of the information representing the cardiac phase contained in the information of the protocol group that is managed and provided by the MRI apparatus 100, the load on the operator in setting a cardiac phase can be reduced.

For example, the MRI apparatus 100 may manage and provide the protocol groups separately according to the purposes of the same type of examinations, for example, examinations for youth and seniors, an examination from a sense of small discomfort, an examination that is a follow-up of a serious cardiac disease. In such a case, for each protocol group, the cardiac phase at which multi-slice views are acquired may be changed even between the same type of protocols.

Generally, the diastole of the heart of a healthy subject is longer than the systole and the systole tends to be longer than the diastole in seniors and patients of serious diseases. Thus, for example, for a cardiac examination, for a protocol group for youth, a cardiac phase is set such that multi-slice views are acquired at diastole and, for a protocol group for seniors, a cardiac phase is set such that multi-slice views are acquired at systole. Thus, according to the purpose of the examination, the cardiac phase at which multi-slice views are acquired can be switched appropriately.

For example, the imaging condition setting unit 133a may acquire attribute information on a subject to be examined or information on a past examination and, on the basis of the acquired information, set a cardiac phase at which multi-slice views are acquired.

For example, when the MRI apparatus 100 is connected to another system that manages information on a patient who is a subject via a network, the imaging condition setting unit 133a acquires the attribute information on the subject to be examined and information on the past examination from the system. The other system is, for example, a hospital information system or a radiology information system, which is referred above.

For example, according to the acquired attribute information on the subject, the imaging condition setting unit 133a sets a cardiac phase at which multi-slice views are acquired. For example, when the subject is at a given age or younger, the imaging condition setting unit 133a sets a cardiac phase such that multi-slice views are acquired at diastole. On the other hand, when the subject is older than the given age, the imaging condition setting unit 133a sets a cardiac phase such that multi-slice views are acquired at systole. Thus, according to the attribute of the subject, the cardiac phase at which multi-slice views are acquired can be switched appropriately.

For example, on the basis of the acquired information on the past examination of the subject, the imaging condition setting unit 133a sets a respiratory phase at which image data is acquired in imaging scan. For example, if multi-slice views are acquired at diastole in the previous examination, the imaging condition setting unit 133a sets a cardiac phase such that multi-slice views are acquired at diastole also in the current examination. On the other hand, if multi-slice views are acquired at systole in the previous examination, the imaging condition setting unit 133a sets a cardiac phase such that multi-slice views are acquired at systole also in the current examination. Thus, according to the state of the examination of the subject, the cardiac phase at which multi-slice views are acquired can be switched appropriately.

(Image Processing System)

For the above-described embodiments, the case has been described where the MRI apparatus 100 that is a medical image diagnosis apparatus performs various types of processing. However, embodiments are not limited to this. For example, an image processing system that includes the MRI apparatus 100 and an image processing apparatus may perform the above-described various types of processing. The image processing apparatus may be, for example, various apparatuses, such as a workstation, a PACS (Picture Archiving and Communications System), an image storage device (image server), a viewer, and an electronic health record system. In this case, for example, the MRI apparatus 100 performs the acquisition performed by the sequence controller 120. On the other hand, the image processing apparatus receives MR data and k-space data that are acquired by the MRI apparatus 100 from the MRI apparatus 100 or an image server via a network, or receives the data that is input by an operator via a recording medium, and stores the data in the storage unit. It is satisfactory if the image processing apparatus performs the above-described various types of processing (such as the processing performed by the image generator 136 and the processing performed by the region deriving unit 133b) on the MR data and the k-space data that are stored in the storage unit.

Program

The instructions represented in the procedure represented in the above-described embodiments can be executed according to a program that is software. A general-purpose computer may store in advance the program and, by reading the program, the same effects as those obtained with the MRI apparatus 100 of the above-described embodiments may be obtained. The instructions described for the above-described embodiments are recorded as a program to be executed by a computer in a magnetic disk (a flexible disk, hard disk, etc.), an optical disk (a CD-RON, CD-R, CD-RW, DVD-ROM, DVD±R, DVD±RW, etc.), a semiconductor memory, or a recording medium similar to this. As long as the storage medium can be read by a computer or an incorporated system, any mode of storage format may be used. A computer reads the program from the recording medium and, according to the program, causes the CPU to execute the instructions described in the program so that the same operations as those of the MRI apparatus 100 of the above-described embodiments can be implemented. In a case where a computer acquires or reads a program, the program may be acquired or read via a network.

Furthermore, an OS (Operating System) that runs on the computer according to the instructions of the program from the recording medium installed in the computer or an incorporated system, database management software, MW (Middleware), such as a network, etc. may perform a part of each process for implementing the above-described embodiments. Furthermore, the storage medium is not limited to media that are independent of the computer or the incorporated system and the storage medium includes a recording medium that downloads the program that is transmitted via a LAN (Local Area Network) or the Internet and stores or temporarily stores the program. Furthermore, the number of recording medium is not limited to one. In a case a process in an embodiment described above is performed from multiple recording mediums, the multiple recording mediums are included in the recording medium described in the embodiment and the configuration of the recording medium may be of any kinds of configuration.

Based on a program recorded in a recording medium, the computer or the installed system in the embodiments are for implementing each process in the above-described embodiments and may have the configuration of any one of a single device, such as a personal computer or a microcomputer, and a system in which multiple devices are connected via a network. The computer of the embodiment is not limited to a personal computer, includes processing units and microcomputers included in processing units, and is a general term of devices and apparatuses that can implement the functions of the embodiments by using a program.

FIG. 21 is a diagram of a hardware configuration of a computer that implements the calculator 130 and the sequence controller 120 according to the embodiments. The calculator 130 and the sequence controller 120 according to the embodiments include, for example, as shown in FIG. 21, a control device, such as a CPU (Central Processing Unit) 210, a storage device, such as a ROM (Read Only Memory) 220 and a RAM (Random Access Memory) 230, a communication I/F 240 that connects to a network and communicates, and a bus 250 that connects these units.

For example, the ROM 220 and the RAM 230 store the program for implementing the processing that is described as one to be performed by the calculator 130 and the sequence controller 120. For example, the program is stored in a computer-readable storage medium, is read from the storage medium, and is stored in the storage device. The CPU 210 reads and executes the program so that the computer is caused to function as the calculator 130 and the sequence controller 120 in the above-described embodiments.

According to the magnetic resonance imaging apparatus according to at least one of the embodiments, multi-slice views can be acquired appropriately.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising:
a controller that performs first imaging for acquiring first image data, that performs second imaging for acquiring second image data and third image data, and that performs third imaging for acquiring fourth image data, the first image data being of a region including a target and a diaphragm, the second imaging and the third imaging being performed with application of motion detection pulses for detecting a respiratory phase, the second image data being of a region including the target at a first respiratory phase, the third image data being of a region including the target at a second respiratory phase being different from the first respiratory phase image; and
a deriving unit that detects a position of the diaphragm from the first image data and that, on a basis of the detected position, derives a region to which the motion detection pulses are applied;
wherein, in the performing of the second imaging, the controller detects the respiratory phase by the application of the motion detection pulses and, on a basis of the detected respiratory phase, controls timings at which the second image data and the third image data are acquired.

2. The magnetic resonance imaging apparatus according to claim 1, wherein the controller acquires both of the second image data and the third image data in a pulse sequence that is executed according to one protocol.

3. The magnetic resonance imaging apparatus according to claim 1, further comprising a generator that generates an image at the first respiratory phase from the second image data and that generates an image at the second respiratory phase from the third image data.

4. The magnetic resonance imaging apparatus according to claim 1, further comprising a setting unit that receives settings of the first respiratory phase and the second respiratory phase,
wherein the controller acquires the second image data and the third image data according to the received settings.

5. The magnetic resonance imaging apparatus according to claim 4, wherein
the setting unit displays, on a display unit, an image at the first respiratory phase generated from the second image data, and an image at the second respiratory phase generated from the third image data, and receives, on at least any one of the images, positioning of the fourth image data that is acquired by performing the third imaging.

6. The magnetic resonance imaging apparatus according to claim 4, wherein
the third imaging is for acquiring the fourth image data at least one of the first respiratory phase and the second respiratory phase, and
in the performing of the third imaging, when acquiring the fourth image data at the first respiratory phase, the controller acquires the fourth image data according to information on a positioning performed by using an image at the first respiratory phase that is generated from the second image data and, when acquiring the fourth image data at the second respiratory phase, the controller acquires the fourth image data according to information on a positioning performed by using an image at the second respiratory phase that is generated from the third image data.

7. The magnetic resonance imaging apparatus according to claim 6, wherein
the setting unit sets any one of the first respiratory phase and the second respiratory phase for a respiratory phase at which the fourth image data is acquired, and
in the performing of the third imaging, the controller acquires the fourth image data at the respiratory phase that is set by the setting unit.

8. The magnetic resonance imaging apparatus according to claim 7, wherein
the setting unit receives, from an operator, an operation for selecting any one of the first respiratory phase and the second respiratory phase and sets a respiratory phase selected by the operation for the respiratory phase at which the fourth image data is acquired.

9. The magnetic resonance imaging apparatus according to claim 7, wherein
the third imaging is for acquiring the fourth image data when a subject is holding breath, at least any one of the first respiratory phase and the second respiratory phase in a state, and
the setting unit receives, from an operator, an operation for selecting which of the first respiratory phase and the second respiratory phase is given to the subject as a notification representing a respiratory phase at which the patient holds the breath in the third imaging and sets the respiratory phase selected by the operation for the respiratory phase at which the fourth image data is acquired.

10. The magnetic resonance imaging apparatus according to claim 7, wherein the setting unit receives, from an operator, an operation for specifying a protocol that is implemented in the third imaging and, according to the protocol specified by the operation, sets the respiratory phase at which the fourth image data is acquired.

11. The magnetic resonance imaging apparatus according to claim 7, wherein the setting unit acquires any one of attribute information and past examination information on a subject to be examined and, according to the acquired information, sets the respiratory phase at which the fourth image data is acquired.

12. The magnetic resonance imaging apparatus according to claim 7, wherein
the third imaging is for sequentially implementing multiple protocols,
the setting unit sets, for each of the multiple protocols, the respiratory phase at which the fourth image data is acquired, and
in the performing of the third imaging, the controller acquires the fourth image data at the respiratory phase that is set by the setting unit according to each of the multiple protocols.

13. The magnetic resonance imaging apparatus according to claim 12, wherein the multiple protocols include a protocol for acquiring the fourth image data in a state of the patient holding breath at least any one of the first respiratory phase and the second respiratory phase.

14. The magnetic resonance imaging apparatus according to claim 12, wherein the multiple protocols include a protocol for acquiring the fourth image data that is imaged under free breathing.

15. The magnetic resonance imaging apparatus according to claim 1, wherein the controller performs the second imaging in synchronization with electrocardiogram and, at a given cardiac phase and when the detected respiratory phase is the first respiratory phase, acquires the second image data and, at the given cardiac phase and when the detected respiratory phase is the second respiratory phase, acquires the third image data.

16. The magnetic resonance imaging apparatus according to claim 15, further comprising a setting unit configured to receive, from an operator, an operation for specifying a protocol that is implemented in the second imaging and, according to the protocol that is specified by the operation, set the given cardiac phase.

17. The magnetic resonance imaging apparatus according to claim 15, further comprising a setting unit that acquires any one of attribute information and past examination information on a patient to be examined and that, according to the acquired information, sets the given cardiac phase.

18. The magnetic resonance imaging apparatus according to claim 1, wherein the deriving unit further detects positions of a top end and a bottom end of a heart from the first image data and, on a basis of the detected positions, derives a region where multi-slice views are imaged.

19. A magnetic resonance imaging apparatus comprising:
a controller that performs first imaging for acquiring first image data, that performs second imaging for sequentially acquiring second image data, and that performs third imaging for acquiring third image data, the first image data being of a region including a target and a diaphragm, the second imaging being performed with application of motion detection pulses for detecting a respiratory phase, the second image data being of a region including the target;
a deriving unit that detects a position of the diaphragm from the first image data and that, on a basis of the detected position, derives a region to which the motion detection pulses are applied; and
a generator that specifies the second image data corresponding to a given respiratory phase from among the sequentially acquired second image data by using the respiratory phase that is detected by the application of the motion detection pulses in the performing of the second imaging and that generates an image by using the specified second image data.
